SEQUENCE LISTING (145 sequences)

SEQ ID NO: 1 - DNA, 1383 bp, Mycobacterium abscessus
acatgcaagtcgaacgggaaaggcccttcggggtactcgagtggcgaacgggtgagtaacacgtgggtgatctgccctgcactctgggataagcctgggaaactgggtctaataccggataggaccacacacttcatggtgagtggtgcaaagcttttgcggtgtgggatgagcccgcggcctatcagcttgttggtggggtaatggcccaccaaggcgacgacgggtagccggcctgagagggtgaccggccacactgggactgagatacggcccagactcctacgggaggcagcagtggggaatattgcacaatgggcgcaagcctgatgcagcgacgccgcgtgagggatgacggccttcgggttgtaaacctctttcagtagggacgaagcgaaagtgacggtacctacagaagaaggaccggccaactacgtgccagcagccgcggtaatacgtagggtccgagcgttgtccggaattactgggcgtaaagagctcgtaggtggtttgtcgcgttgttcgtgaaaactcacagcttaactgtgggcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtgggtagcgaacaggattagataccctggtagtccacgccgtaaacggtgggtactaggtgtgggtttccttccttgggatccgtgccgtagctaacgcattaagtaccccgcctggggagtacggtcgcaagactaaaactcaaaggaattgacgggggcccgcacaagcggcggagcatgtggattaattcgatgcaacgcgaagaaccttacctgggtttgacatgcacaggacgtatctagagataggtattcccttgtggcctgtgtgcaggtggtgcatggctgtcgtcagctcgtgtcgtgagatgttgggttaagtcccgcaacgagcgcaacccttgtcctatgttgccagcgggtaatgccggggactcgtaggagactgccggggtcaactcggaggaaggtggggatgacgtcaagtcatcatgccccttatgtccagggcttcacacatgctacaatggccagtacagagggctgcgaagccgtaaggtggagcgaatcccttaaagctggtctcagttcggattggggtctgcaactcgaccccatgaagtcggagtcgctagtaatcgcagatcagcaacgctgcggtgaatacgttcccgggccttgtacacaccgcccgtcacgtcatgaaagtcggtaacacccgaagccagtggcctaaccttttggaggga

SEQ ID NO: 2 - DNA, 1454 bp, Mycobacterium avium
gacgaacgctggcggcgtgcttaacacatgcaagtcgaacggaaaggcctcttcggaggtactcgagtggcgaacgggtgagtaacacgtgggcaatctgccctgcacttcgggataagcctgggaaactgggtctaataccggataggacctcaagacgcatgtcttctggtggaaagcttttgcggtgtgggatgggcccgcggcctatcagcttgttggtggggtgacggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacctctttcaccatcgacgaaggtccgggttttctcggattgacggtaggtggagaagaagcaccggccaactacgtgccagcagccgcggtaatacgtagggtgcgagcgttgtccggaattactgggcgtaaagagctcgtaggtggtttgtcgcgttgttcgtgaaatctcacggcttaactgtgagcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgcgtctagagataggcgttcccttgtggcctgtgtgcaggtggtgcatggctgtcgtcagctcgtgtcgtgagatgttgggttaagtcccgcaacgagcgcaacccttgtctcatgttgccagcgggtaatgccggggactcgtgagagactgccggggtcaactcggaggaaggtggggatgacgtcaagtcatcatgccccttatgtccagggcttcacacatgctacaatggccggtacaaagggctgcgatgccgtaaggttaagcgaatccttttaaagccggtctcagttcggattggggtctgcaactcgaccccatgaagtcggagtcgctagtaatcgcagatcagcaacgctgcggtgaatacgttcccgggccttgtacacaccgcccgtcacgtcatgaaagtcggtaacacccgaagccagtggcctaacccttttgggagggagctgtcgaaggtgggatcggcgattgggac

SEQ ID NO: 3 - DNA, 1421 bp, Mycobacterium bovis
ggcggcgtgcttaacacatgcaagtcgaacggaaaggtctcttcggagatactcgagtggcgaacgggtgagtaacacgtgggtgatctgccctgcacttcgggataagcctgggaaactgggtctaataccggataggaccacgggatgcatgtcttgtggtggaaagcgctttagcggtgtgggatgagcccgcggcctatcagcttgttggtggggtgacggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacctctttcaccatcgacgaaggtccgggttctctcggattgacggtaggtggagaagaagcaccggccaactacgtgccagcagccgcggtaatacgtagggtgcgagcgttgtccggaattactgggcgtaaagagctcgtaggtggtttgtcgcgttgttcgtgaaatctcacggcttaactgtgagcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgcgtctagagataggcgttcccttgtggcctgtgtgcaggtggtgcatggctgtcgtcagctcgtgtcgtgagatgttgggttaagtcccgcaacgagcgcaacccttgtctcatgttgccagcacgtaatggtggggactcgtgagagactgccggggtcaactcggaggaaggtggggatgacgtcaagtcatcatgccccttatgtccagggcttcacacatgctacaatggccggtacaaagggctgcgatgccgcgaggttaagcgaatccttaaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaacccttgggagggagctgtcga

SEQ ID NO: 4 - DNA, 1439 bp, Mycobacterium chelonae
gacgaacgctggcggcgtgcttaacacatgcaagtcgaacgggaaaggcccttcggggtactcgagtggcgaacgggtgagtaacacgtgggtgatctgccctgcactctgggataagcctgggaaactgggtctaataccggataggaccacacacttcatggtgagtggtgcaaagcttttgcggtgtgggatgagcccgcggcctatcagcttgttggtggggtaatggcccaccaaggcgacgacgggtagccggcctgagagggtgaccggccacactgggactgagatacggcccagactcctacgggaggcagcagtggggaatattgcacaatgggcgcaagcctgatgcagcgacgccgcgtgagggatgacggccttcgggttgtaaacctctttcagtagggacgaagcgaaagtgacggtacctacagaagaaggaccggccaactacgtgccagcagccgcggtaatacgtagggtccgagcgttgtccggaattactgggcgtaaagagctcgtaggtggtttgtcgcgttgttcgtgaaaactcacagcttaactgtgggcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtgggtagcgaacaggattagataccctggtagtccacgccgtaaacggtgggtactaggtgtgggtttccttccttgggatccgtgccgtagctaacgcattaagtaccccgcctggggagtacggtcgcaagactaaaactcaaaggaattgacgggggcccgcacaagcggcggagcatgtggattaattcgatgcaacgcgaagaaccttacctgggtttgacatgcgcaggacgtatctagagataggtattcccttgtggcctgcgtgcaggtggtgcatggctgtcgtcagctcgtgtcgtgagatgttgggttaagtcccgcaacgagcgcaacccttgtcctatgttgccagcgggtaatgccggggactcgtaggagactgccggggtcaactcggaggaaggtggggatgacgtcaagtcatcatgccccttatgtccaggctttcacacatgctacaatggccagtacagagggctgcgaagccgcaaggtggagcgaatcccttaaagctggtctcagttcggattggggtctgcaactcgaccccatgaagtcggagtcgctagtaatcgcagatcagcaacgctgcggtgaatacgttcccgggccttgtacacaccgcccgtcmcgtcatgaaagtcggtaacacccgaagccagtggcctaaccttttggagggagctgtcgaaggtgggatcggcgattgggacg

SEQ ID NO: 5 - DNA, 1482 bp, Mycobacterium farcinogenes
cgaacgctcgcggcgtgcttaacacatgcaagtcgaacggaaaggcccttcggggtactcgagtggcgaacgggtgagtaacacgtgggtgatctgccctgcactttgggataagcctgggaaactgggtctaataccggataggaccacgcgcttcatggtgtgtggtggaaagcttttgcggtgtgggatgggcccgcggcctatcagcttgttggtggggtaatggcctaccaaggcgacgacgggtagccggcctgagagggtgaccggccacactgggactgagatacggcccagactcctacgggaggcagcagtggggaatattgcacaatgggcgcaagcctgatgcagcgacgccgcgtgagggatgacggccttcgggttgtaaacctctttcaatagggacgaagcgcaagtgacggtacctatagaagaaggaccggccaactacgtgccagcagccgcggtaatacgtagggtccgagcgttgtccggaattactgggcgtaaagagctcgtaggtggtttgtcgcgttgttcgtgaaaactcacagcttaactgtgggcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgccagtagagatattggttcccttgtggcctgtgtgcaggtggtgcatggctgtcgtcagctcgtgtcgtgagatgttgggttaagtcccgcaacgagcgcaacccttgtctcatgttgccagcacgttatggtggggactcgtgagagactgccggggtcaactcggaggaaggtggggatgacgtcaagtcatcatgccccttatgtccagggcttcacacatgctacaatggccggtacaaagggctgcgatgccgtgaggtggagcgaatcctttcaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccttgtggagggagccgtcgaaggtgggatcggcgattgggacgaagtcgtaacaaggtagccgtaccggaaggtgcggctggatcac

SEQ ID NO: 6 - DNA, 1449 bp, Mycobacterium fortuitum
ggcggcgtgcttaacacatgcaagtcgaacggaaaggcccttcgggtactcgagtggcgaacgggtgagtaacacgtgggtgatctgccctgcactttgggataagcctgggaaactgggtctaataccgaatatgaccgcgcacttcctggtgtgtggtggaaagcttttgcggtgtgggatgggcccgcggcctatcagcttgttggtggggtaatggcctaccaaggcgacgacgggtagccggcctgagagggtgaccggccacactgggactgagatacggcccagactcctacgggaggcagcagtggggaatattgcacaatgggcgcaagcctgatgcagcgacgccgcgtgagggatgacggccttcgggttgtaaacctctttcaatagggacgaagcgcaagtgacggtacctatagaagaaggaccggccaactacgtgccagcagccgcggtaatacgtagggtccgagcgttgtccggaattactgggcgtaaagagctcgtaggtggtttgtcgcgttgttcgtgaaaactcacagcttaactgtgggcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacggcgtaaacggtgggtactaggtgtgggtttccttccttgggatccgtgccgtagctaacgcattaagtaccccgcctggggagtacggccgcaaggctaaaactcaaagaaattgacggggggccgcacaagcggcggagcatgtggattaattcgatgcaacgcgaagaaccttacctgggtttgacatgcacaggacgccagtagagatattggttcccttgtggcctgtgtgcaggtggtgcatggctgtcgtcagctcgtgtcgtgagatgttgggttaagtcccgcaacgagcgcaacccttatcttatgttgccagcgcgtaatggcggggactcgtgagagactgccggggtcaactcggaggaaggtggggatgacgtcaagtcatcatgccccttatgtccagggcttcacacatgctacaatggccggtacaaagggctgcgatgccgtgaggtggagcgaatcctttcaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccttgtggagggagccgtcgaaggtgggatcggcgattgggacgaagtcgtaacaaggtagccg

SEQ ID NO: 7 - DNA, 1461 bp, Mycobacterium gordonae
ggcggcgtgcttaacacatgcaagtcgaacggtaaggcccttcggggtacacgagtggcgaacgggtgagtaacacgtgggtaatctgccctgcacatcgggataagcctgggaaactgggtctaataccgaataggaccacaggacacatgtcctgtggtggaaagcttttgcggtgtgggatgggcccgcggcctatcagcttgttggtggggtgatggcctaccaaggcgacgacgggtagccggcctgagagggtgtccggccacactgggactgagatacggcccagactcctacgggaggcagcagtggggaatattgcacaatgggcgaaagcctgatgcagcgacgccgcgtgggggatgacggccttcgggttgtaaacctctttcaccatcgacgaaggtccgggttttctcgggctgacggtaggtggagaagaagcaccggccaactacgtgccagcagccgcggtaatacgtagggtgcgagcgttgtccggaattactgggcgtaaagagctcgtaggtggtttgtcgcgttgttcgtgaaatctcacggcttaactgtgagcgtgcgggcgatacgggcagacttgagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaagaaattgacgggggcccgcacaagcggcggagcatgtggattaattcgatgcaacgcgaagaaccttacctgggtttgacatgcacaggacgccggcagagatgtcggttcccttgtggcctgtgtgcaggtggtgcatgcctgtcgtcagctcgtgtcgtgagatgttgggttaagtcccgcaacgagcgcaacccttgtctcatgttgccagcgggtaatgccggggactcgtgagagactgccggggtcaactcggaggaaggtggggatgacgtcaagtcatcatgccccttatgtccagggcttcacacatgctacaatggccggtacaaagggctgcgatgccgcgaggttaagcgaatccttttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaacctttgggagggagctgtcgaaggtgggatcggcgattgggacgaagtcgtaacaaggtagccg

SEQ ID NO: 8 - DNA, 1527 bp, Mycobacterium heckeshornense
tgatcctggctcaggacgaacgctggcggcgtgcttaacacatgcaagtcgaacggaaaggcccgcttcggtgggtgctcgagtggcgaacgggtgagtaacacgtgggtgacctgccctgcacttcgggataagcctgggaaactgggtctaataccggataggaccgcgccatgcatgtggtgtggtggaaagcgtgtggtagtggtgtgggatgggcccgcggcctatcagcttgttggtggggtgatggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacctctttcaccatcgacgaagccgcagcttttgttgtggtgacggtaggtggagaagaagcaccggccaactacgtgccagcagccgcggtaatacgtagggtgcaagcgttgtccggaattactgggcgtaaagagctcgtaggcggcttgtcgcgttgttcgtggaatgccacagcttaactgtgggcgtgcgggcgatacgggcaggctggagtgctgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggttctttcctgaaggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgcgtctagagataggcgttcccttgtggcctgtgtgcaggtggtgcatggctgtcgtcagctcgtgtcgtgagatgttgggttaagtcccgcaacgagcgcaacccttgtcccatgttgccagcacgtgatggtggggactcatgggagactgccggggtcaactcggaggaaggtggggatgacgtcaagtcatcatgccccttatgtccagggcttcacacatgctacaatggccggtacaaagggctgcgatgccgtgaggttaagcgaatccttgtaaagccggtctcagttcggatcggggtctgcaactcgaccccgtgaagtcggagtcgctagtaatcgcagatcagcaatgctgcggtgaatacgttcccgggccttgtacacaccgcccgtcacgtcatgaaagtcggtaacacccgaagcccatggcccaacccgtttgggagggagtggtcgaaggtgggatcggcgattgggacgaagtcgtaacaaggtagccgtaccggaaggtgcggctggatcacctccttaa

SEQ ID NO: 9 - DNA, 1452 bp, Mycobacterium intracellulare
ttaacacatgcaagtggaacggaaaggccccttcggggtactcgagtggcgaacgggtgagtaacacgtgggcaatctgccctgcacttcgggataagcctgggaaactgggtctaataccggataggacctttaggcgcatgtctttaggtggaaagcttttgcggtgtgggatgggcccgcggcctatcagcttgttggtggggtgatggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacctctttcaccatcgacgaaggtccgggttttctcggattgacggtaggtggagaagaagcaccggccaactacgtgccagcagccgcggtaatacgtagggtgcgagcgttgtccggaattactgggcgtaaagagctcgtaggtggtttgtcgcgttgttcgtgaaatctcacggcttaactgtgagcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaaggaattgacgggggccggcacaagcggcggagcatgtggattaattcgatgcaacgcgaagaaccttacctgggtttgacatgcacaggacgcgtctagagataggcgttcccttgtggcctgtgtgcaggtggtgcatggctgtcgtcagctcgtgtcgtgagatgttgggttaagtcccgcaacgagcgcaacccttgtctcatgttgccagcgggtaatgccggggactcgtgagagactgccggggtcaactcggaggaaggtggggatgacgtcaagtcatcatgccccttatgtccagggcttcacacatgctacaatggccggtacaaagggctgcgatgccgcaaggttaagcgaatccttttaaagccggtctcagttcggattggggtctgcaactcgaccccatgaagtcggagtcgctagtaatcgcagatcagcaacgctgcggtgaatacgttcccgggccttgtacacaccgcccgtcacgtcatgaaagtcggtaacacccgaagccagtggcctaacccttgggagggagctgtcgaaggtgggatcggcgattgggacgaagtcgtaacaaggtagccg

SEQ ID NO: 10 - DNA, 1463 bp, Mycobacterium kansasii
gcggcgtgcttaacacatgcaagtcgaacggaaaggtctcttcggagacactcgagtggcgaacgggtgagtaacacgtgggcaatctgccctgcacaccgggataagcctgggaaactgggtctaataccggataggaccacttggcgcatgccttgtggtggaaagcttttgcggtgtgggatgggcccgcggcctatcagcttgttggtggggtgacggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacctctttcaccatcgacgaaggtccgggttctctcggattgacggtaggtggagaagaagcaccggccaactacgtgccagcagccgcggtaatacgtagggtgcgagcgttgtccggaattactgggcgtaaagagctcgtaggtggtttgtcgcgttgttcgtgaaatctcacggcttaactgtgagcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgcgtctagagataggcgttcccttgtggcctgtgtgcaggtggtgcatggctgtcgtcagctcgtgtcgtgagatgttgggttaagtcccgcaacgagcgcaacccttgtctcatgttgccagcgggtaatgccggggactcgtgagagactgccggggtcaactcggaggaaggtggggatgacgtcaagtcatcatgccccttatgtccagggcttcacacatgctacaatggccggtacaaagggctgcgatgccgcgaggttaagcgaatccttttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaaccctcgggagggagctgtcgaaggtgggatcggcgattgggacgaagtcgtaacaaggtagccgt

SEQ ID NO: 11 - DNA, 1321 bp, Mycobacterium kubicae
gtgcttaacacatgcaagtcgaacggaaaggccccttcgggggtactcgagtggcgaacgggtgagtaacacgtgggtgatctaccctgcacttcgggataagcctgggaaactgggtctaataccggataggaccatgagatgcatgtcttatggtggaaagcttttgcggtgtgggatgggcccgcggcctatcagcttgttggtggggtgacggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacctctttcagcagggacgaagcgcaagtgacggtacctgcagaagaagcaccggccaactacgtgccagcagccgcggtaatacgtagggtgcgagcgttgtccggaattactgggcgtaaagagctcgtaggtggtttgtcgcgttgttcgtgaaaaccgggggcttaaccctcggcgtgcgggcgatacgggcagactggagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgcgtctagagataggcgttcccttgtggcctgtgtgcaggtggtgcatggctgtcgtcagctcgtgtcgtgagatgttgggttaagtcccgcaacgagcgcaacccttgtctcatgttgccagcgggtaatgccggggactcgtgagagactgccggggtcaactcggaggaaggtggggatgacgtcaagtcatcatgccccttatgtccagggcttcacacatgctacaatggccggtacaaagggctgcgatgccgcgaggttaagcgaatccttttaaagccggtctcagttcggatcggggtctgcaactcgaccccgtgaagtcggagtcgctagtaatcgcagatcagcaacgctgcggtgaatacgttcccggg

SEQ ID NO: 12 - DNA, 1421 bp, Mycobacterium lentiflavum
ggcggcgtgcttaacacatgcaagtcgaacggaaaggcctcttcggaggtactcgagtggcgaacgggtgagtaacacgtgggtaatctgccctgcacttcgggataagcctgggaaactgggtctaataccggataggaccttttggcgcatgccttttggtggaaagcttttgcggtgtgggatgggcccgcggcctatcagcttgttggtggggtgacggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacctctttcagcagggacgaagcgcaagtgacggtacctgcagaagaagcaccggccaactacgtgccagcagccgcggtaatacgtagggtgcgagcgttgtccggaattactgggcgtaaagagctcgtaggtggtttgtcgcgttgttcgtgaaaaccgggggcttaaccctcggcgtgcgggcgatacgggcagactggagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttggaatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgccggcagagatgtcggttcccttgtggcctgtgtgcaggtggtgcatggctgtcgtcagctcgtgtcgtgagatgttgggttaagtcccgcaacgagcgcaacccttgtctcatgttgccagcgcgtaatggcggggactcgtgagagactgccggggtcaactcggaggaaggtggggatgacgtcaagtcatcatgccccttatgtccagggcttcacacatgctacaatggccggtacaaagggctgcgatgccgtaaggttaagcgaatccttttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaaccttttggagggagctgtcgaaggtgggatcggc

SEQ ID NO: 13 - DNA, 1455 bp, Mycobacterium mucogenicum
gacgaacgctggcggcgtgcttaacacatgcaagtcgaacggaaaggcccttcggggtactcgagtggcgaacgggtgagtaacacgtgggtgatctgccctgcactttgggataagcctgggaaactgggtctaataccgaataggaccacgcgcttcatggtgtgtggtggaaagcttttgcggtgtgggatgggcccgcggcctatcagcttgttggtggggtaatggcctaccaaggcgacgacgggtagccggcctgagagggtgaccggccacactgggactgagatacggcccagactcctacgggaggcagcagtggggaatattgcacaatgggcgcaagcctgatgcagcgacgccgcgtgagggatgacggccttcgggttgtaaacctctttcaatagggacgaagcgcaagtgacggtacctatagaagaagcaccggccaactacgtgccagcagccgcggtaatacgtagggtgcgagcgttgtccggaattactgggcgtaaagagctcgtaggtggtttgtcgcgttgttcgtgaaaactcacagcttaactgtgggcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggttccttccttgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgccggcagagatgtcggttcccttgtggcctgtgtgcaggtggtgcatggctgtcgtcagctcgtgtcgtgagatgttgggttaagtcccgcaacgagcgcaacccttgtcctatgttgccagcgggttatgccggggactcgtaggagactgccggggtcaactcggaggaaggtggggatgacgtcaagtcatcatgccccttatgtccagggcttcacacatgctacaatggccggtacaaagggctgcgatgccgtgaggtggagcgaatcctttcaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccttgtggagggagccgtcgaaggtgggatcggcgattgggacgaagtcgtaacaaggta

SEQ ID NO: 14 - DNA, 1415 bp, Mycobacterium paraffinicum
cgtgcttaacacatgcaagtcgaacggaaaggccccttcgggggtactcgagtggcgaacgggtgagtaacacgtgggcaatctgccctgcacttcgggataagcctgggaaactgggtctaataccggataggaccacttggcgcatgccttgtggtggaaagcttttgcggtgtgggatgggcccgcggcctatcagcttgttggtggggtgatggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacctctttcaccatcgacgaaggctcacttcgtgagttgacggtaggtggagaagaagcaccggccaactacgtgccagcagccgcggtaatacgtagggtgcgagcgttgtccggaattactgggcgtaaagagctcgtaggtggtttgtcgcgttgttcgtgaaatctcacggcttaactgtgagcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgcgtctagagataggcgttcccttgtggcctgtgtgcaggtggtgcatggctgtcgtcagctcgtgtcgtgagatgttgggttaagtcccgcaacgagcgcaacccttgtctcatgttgccagcgggtaatgccggggactcgtgagagactgccggggtcaactcggaggaaggtggggatgacgtcaagtcatcatgccccttatgtccagggcttcacacatgctacaatggccggtacaaagggctgcgatgccgcaaggttaagcgaatccttttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaacccttgggagggagctgtcgaagg

SEQ ID NO: 15 - DNA, 1484 bp, Mycobacterium simiae
ggcggcgtgcttaacacatgcaagtcgaacggaaaggccccttcgggggtactcgagtggcgaacgggtgagtaacacgtgggtaatctgccctgcacttcgggataagcctgggaaactgggtctaataccggataggaccacttggcgcatgccttgtggtggaaagcttttgcggtgtgggatgggcccgcggcctatcagcttgttggtggggtgacggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacctctttcagcagggacgaagcgcaagtgacggtacctgcagaagaagcaccggccaactacgtgccagcagccgcggtaatacgtagggtgcgagcgttgtccggaattactgggcgtaaagagctcgtaggtggtttgtcgcgttgttcgtgaaaaccgggggcttaaccctcggcgtgcgggcgatacgggcagactggagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttggaatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgccggcagagatgtcggttcccttgtggcctgtgtgcaggtggtgcatggctgtcgtcagctcgtgtcgtgagatgttgggttaagtcccgcaacgagcgcaacccttgtctcatgttgccagcgggtaatgccggggactcgtgagagactgccggggtcaactcggaggaaggtggggatgacgtcaagtcatcatgccccttatgtccagggcttcacacatgctacaatggccggtacaaagggctgcgatgccgcaaggttaagcgaatccttttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaaccttttggagggagctgtcgaaggtgggatcggcgattgggacgaagtcgtaacaaggtagccgtaccggaaggtgcggctggatcacctcctttct

SEQ ID NO: 16 - DNA, 1462 bp, Mycobacterium szulgai
ggcggcgtgcttaacacatgcaagtcgaacggaaaggccccttcggggtactcgagtggcgaacgggtgagtaacacgtgggtaatctgccctgcacttcgggataagcctgggaaactgggtctaataccggataggaccccgaggcgcatgccttggggtggaaagcttttgcggtgtgggatgggcccgcggcctatcagcttgttggtggggtgacggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacctctttcaccatcgacgaaggtccgggttttctcggattgacggtaggtggagaagaagcaccggccaactacgtgccagcagccgcggtaatacgtagggtgcgagcgttgtccggaattactgggcgtaaagagctcgtaggtggtttgtcgcgttgttcgtgaaatctcacggcttaactgtgagcgtgcgggcgatacgggcagactggagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacggcgtaaacggtgggtactaggtgtgggtttccttccttgggatccgtgccgtagctaacgcattaagtaccccgcctggggagtacggcggcaaggctaaaactcaaaggaattgacgggggcccgcacaagcggcggagcatgtggattaattcgatgcaacgcgaagaaccttacctgggtttgacatgcacaggacgcgtctagagataggcgttcccttgtggcctgtgtgcaggtggtgcatggctgtcgtcagctcgtgtcgtgagatgttgggttaagtcccgcaacgagcgcaacccttgtctcatgttgccagcgggtaatgccggggactcgtgagagactgccggggtcaactcggaggaaggtggggatgacgtcaagtcatcatgccccttatgtccagggcttcacacatgctacaatggccggtacaaagggctgcgatgccgcgaggttaagcgaatccttttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaacccttgggagggagctgtcgaaggtgggatcggcgattgggacgaagtcgtaacaaggtagccg

SEQ ID NO: 17 - DNA, 1416 bp, Mycobacterium tuberculosis
ggcggcgtgcttaacacatgcaagtcgaacggaaaggtctcttcggagatactcgagtggcgaacgggtgagtaacacgtgggtgatctgccctgcacttcgggataagcctgggaaactgggtctaataccggataggaccacgggatgcatgtcttctggtggaaagcgctttagcggtgtgggatgagcccgcggcctatcagcttgttggtggggtgacggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacctctttcaccatcgacgaaggtccgggttctctcggattgacggtaggtggagaagaagcaccggccaactacgtgccagcagccgcggtaatacgtagggtgcgagcgttgtccggaattactgggcgtaaagagctcgtaggtggtttgtcgcgttgttcgtgaaatctcacggcttaactgtgagcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgcgtctagagataggcgttcccttgtggcctgtgtgcaggtggtgcatggctgtcgtcagctcgtgtcgtgagatgttgggttaagtcccgcaacgagcgcaacccttgtctcatgttgccagcacgtaatggtggggactcgtgagagactgccggggtcaactcggaggaaggtggggatgacgtcaagtcatcatgccccttatgtccagggcttcacacatgctacaatggccggtacaaagggctgcgatgccgcgaggttaagcgaatccttaaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaacccttgggagggagct

SEQ ID NO: 18 - DNA, 15 bp, Artificial Sequence
  primer
taacacatgcaagtc

SEQ ID NO: 19 - DNA, 16 bp, Artificial Sequence
  primer
ttaacacatgcaagtc

SEQ ID NO: 20 - DNA, 17 bp, Artificial Sequence
  primer
cttaacacatgcaagtc

SEQ ID NO: 21 - DNA, 18 bp, Artificial Sequence
  primer
gcttaacacatgcaagtc

SEQ ID NO: 22 - DNA, 17 bp, Artificial Sequence
  primer
gcttaacacatgcaagt

SEQ ID NO: 23 - DNA, 16 bp, Artificial Sequence
  primer
gcttaacacatgcaag

SEQ ID NO: 24 - DNA, 15 bp, Artificial Sequence
  primer
gcttaacacatgcaa

SEQ ID NO: 25 - DNA, 15 bp, Artificial Sequence
  primer
tgcttaacacatgca

SEQ ID NO: 26 - DNA, 16 bp, Artificial Sequence
  primer
tgcttaacacatgcaa

SEQ ID NO: 27 - DNA, 17 bp, Artificial Sequence
  primer
tgcttaacacatgcaag

SEQ ID NO: 28 - DNA, 18 bp, Artificial Sequence
  primer
tgcttaacacatgcaagt

SEQ ID NO: 29 - DNA, 19 bp, Artificial Sequence
  primer
tgcttaacacatgcaagtc

SEQ ID NO: 30 - DNA, 20 bp, Artificial Sequence
  primer
gtgcttaacacatgcaagtc

SEQ ID NO: 31 - DNA, 19 bp, Artificial Sequence
  primer
gtgcttaacacatgcaagt

SEQ ID NO: 32 - DNA, 18 bp, Artificial Sequence
  primer
gtgcttaacacatgcaag

SEQ ID NO: 33 - DNA, 17 bp, Artificial Sequence
  primer
gtgcttaacacatgcaa

SEQ ID NO: 34 - DNA, 16 bp, Artificial Sequence
  primer
gtgcttaacacatgca

SEQ ID NO: 35 - DNA, 15 bp, Artificial Sequence
  primer
cgtgcttaacacatg

SEQ ID NO: 36 - DNA, 16 bp, Artificial Sequence
  primer
cgtgcttaacacatgc

SEQ ID NO: 37 - DNA, 17 bp, Artificial Sequence
  primer
cgtgcttaacacatgca

SEQ ID NO: 38 - DNA, 18 bp, Artificial Sequence
  primer
cgtgcttaacacatgcaa

SEQ ID NO: 39 - DNA, 19 bp, Artificial Sequence
  primer
cgtgcttaacacatgcaag

SEQ ID NO: 40 - DNA, 20 bp, Artificial Sequence
  primer
cgtgcttaacacatgcaagt

SEQ ID NO: 41 - DNA, 21 bp, Artificial Sequence
  primer
cgtgcttaacacatgcaagtc

SEQ ID NO: 42 - DNA, 22 bp, Artificial Sequence
  primer
gcgtgcttaacacatgcaagtc

SEQ ID NO: 43 - DNA, 21 bp, Artificial Sequence
  primer
gcgtgcttaacacatgcaagt

SEQ ID NO: 44 - DNA, 20 bp, Artificial Sequence
  primer
gcgtgcttaacacatgcaag

SEQ ID NO: 45 - DNA, 19 bp, Artificial Sequence
  primer
gcgtgcttaacacatgcaa

SEQ ID NO: 46 - DNA, 18 bp, Artificial Sequence
  primer
gcgtgcttaacacatgca

SEQ ID NO: 47 - DNA, 17 bp, Artificial Sequence
  primer
gcgtgcttaacacatgc

SEQ ID NO: 48 - DNA, 16 bp, Artificial Sequence
  primer
gcgtgcttaacacatg

SEQ ID NO: 49 - DNA, 15 bp, Artificial Sequence
  primer
gcgtgcttaacacat

SEQ ID NO: 50 - DNA, 15 bp, Artificial Sequence
  primer
ttaacacatgcaagt

SEQ ID NO: 51 - DNA, 15 bp, Artificial Sequence
  primer
cttaacacatgcaag

SEQ ID NO: 52 - DNA, 16 bp, Artificial Sequence
  primer
cttaacacatgcaagt

SEQ ID NO: 53 - DNA, 15 bp, Artificial Sequence
  primer
gtgcttaacacatgc

SEQ ID NO: 54 - DNA, 15 bp, Artificial Sequence
  primer
gatatctgcgcattc

SEQ ID NO: 55 - DNA, 16 bp, Artificial Sequence
  primer
tgatatctgcgcattc

SEQ ID NO: 56 - DNA, 15 bp, Artificial Sequence
  primer
tgatatctgcgcatt

SEQ ID NO: 57 - DNA, 15 bp, Artificial Sequence
  primer
ctgatatctgcgcat

SEQ ID NO: 58 - DNA, 16 bp, Artificial Sequence
  primer
ctgatatctgcgcatt

SEQ ID NO: 59 - DNA, 17 bp, Artificial Sequence
  primer
ctgatatctgcgcattc

SEQ ID NO: 60 - DNA, 18 bp, Artificial Sequence
  primer
cctgatatctgcgcattc

SEQ ID NO: 61 - DNA, 17 bp, Artificial Sequence
  primer
cctgatatctgcgcatt

SEQ ID NO: 62 - DNA, 16 bp, Artificial Sequence
  primer
cctgatatctgcgcat

SEQ ID NO: 63 - DNA, 15 bp, Artificial Sequence
  primer
cctgatatctgcgca

SEQ ID NO: 64 - DNA, 15 bp, Artificial Sequence
  primer
tcctgatatctgcgc

SEQ ID NO: 65 - DNA, 16 bp, Artificial Sequence
  primer
tcctgatatctgcgca

SEQ ID NO: 66 - DNA, 17 bp, Artificial Sequence
  primer
tcctgatatctgcgcat

SEQ ID NO: 67 - DNA, 18 bp, Artificial Sequence
  primer
tcctgatatctgcgcatt

SEQ ID NO: 68 - DNA, 19 bp, Artificial Sequence
  primer
tcctgatatctgcgcattc

SEQ ID NO: 69 - DNA, 20 bp, Artificial Sequence
  primer
ctcctgatatctgcgcattc

SEQ ID NO: 70 - DNA, 19 bp, Artificial Sequence
  primer
ctcctgatatctgcgcatt

SEQ ID NO: 71 - DNA, 18 bp, Artificial Sequence
  primer
ctcctgatatctgcgcat

SEQ ID NO: 72 - DNA, 17 bp, Artificial Sequence
  primer
ctcctgatatctgagca

SEQ ID NO: 73 - DNA, 16 bp, Artificial Sequence
  primer
ctcctgatatctgcgc

SEQ ID NO: 74 - DNA, 15 bp, Artificial Sequence
  primer
ctcctgatatctgcg

SEQ ID NO: 75 - DNA, 15 bp, Artificial Sequence
  primer
cctcctgatatctgc

SEQ ID NO: 76 - DNA, 16 bp, Artificial Sequence
  primer
cctcctgatatctgcg

SEQ ID NO: 77 - DNA, 17 bp, Artificial Sequence
  primer
cctcctgatatctgcgc

SEQ ID NO: 78 - DNA, 18 bp, Artificial Sequence
  primer
cctcctgatatctgcgca

SEQ ID NO: 79 - DNA, 19 bp, Artificial Sequence
  primer
cctcctgatatctgcgcat

SEQ ID NO: 80 - DNA, 20 bp, Artificial Sequence
  primer
cctcctgatatctgcgcatt

SEQ ID NO: 81 - DNA, 21 bp, Artificial Sequence
  primer
cctcctgatatctgcgcattc

SEQ ID NO: 82 - DNA, 22 bp, Artificial Sequence
  primer
tcctcctgatatctgcgcattc

SEQ ID NO: 83 - DNA, 21 bp, Artificial Sequence
  primer
tcctcctgatatctgcgcatt

SEQ ID NO: 84 - DNA, 20 bp, Artificial Sequence
  primer
tcctcctgatatctgcgcat

SEQ ID NO: 85 - DNA, 19 bp, Artificial Sequence
  primer
tcctcctgattactgcgca

SEQ ID NO: 86 - DNA, 18 bp, Artificial Sequence
  primer
tcctcctgatatctgcgc

SEQ ID NO: 87 - DNA, 17 bp, Artificial Sequence
  primer
tcctcctgatatctgcg

SEQ ID NO: 88 - DNA, 16 bp, Artificial Sequence
  primer
tcctcctgatatctgc

SEQ ID NO: 89 - DNA, 15 bp, Artificial Sequence
  primer
tcctcctgatatctg

SEQ ID NO: 90 - DNA, 15 bp, Artificial Sequence
  primer
cagccgcggtaatac

SEQ ID NO: 91 - DNA, 16 bp, Artificial Sequence
  primer
gcagccgcggtaatac

SEQ ID NO: 92 - DNA, 15 bp, Artificial Sequence
  primer
gcagccgcggtaata

SEQ ID NO: 93 - DNA, 16 bp, Artificial Sequence
  primer
agcagccgcggtaata

SEQ ID NO: 94 - DNA, 17 bp, Artificial Sequence
  primer
agcagccgcggtaatac

SEQ ID NO: 95 - DNA, 15 bp, Artificial Sequence
  primer
agcagccgcggtaat

SEQ ID NO: 96 - DNA, 15 bp, Artificial Sequence
  primer
cagcagccgcggtaa

SEQ ID NO: 97 - DNA, 16 bp, Artificial Sequence
  primer
cagcagccgcggtaat

SEQ ID NO: 98 - DNA, 17 bp, Artificial Sequence
  primer
cagcagccgcggtaata

SEQ ID NO: 99 - DNA, 18 bp, Artificial Sequence
  primer
cagcagccgcggtaatac

SEQ ID NO: 100 - DNA, 19 bp, Artificial Sequence
  primer
ccagcagccgcggtaatac

SEQ ID NO: 101 - DNA, 18 bp, Artificial Sequence
  primer
ccagcagccgcggtaata

SEQ ID NO: 102 - DNA, 17 bp, Artificial Sequence
  primer
ccagcagccgcggtaat

SEQ ID NO: 103 - DNA, 16 bp, Artificial Sequence
  primer
ccagcagccgcggtaa

SEQ ID NO: 104 - DNA, 15 bp, Artificial Sequence
  primer
ccagcagccgcggta

SEQ ID NO: 105 - DNA, 15 bp, Artificial Sequence
  primer
gccagcagccgcggt

SEQ ID NO: 106 - DNA, 16 bp, Artificial Sequence
  primer
gccagcagccgcggta

SEQ ID NO: 107 - DNA, 17 bp, Artificial Sequence
  primer
gccagcagccgcggtaa

SEQ ID NO: 108 - DNA, 18 bp, Artificial Sequence
  primer
gccagcagccgcggtaat

SEQ ID NO: 109 - DNA, 19 bp, Artificial Sequence
  primer
gccagcagccgcggtaata

SEQ ID NO: 110 - DNA, 20 bp, Artificial Sequence
  primer
gccagcagccgcggtaatac

SEQ ID NO: 111 - DNA, 21 bp, Artificial Sequence
  primer
tgccagcagccgcggtaatac

SEQ ID NO: 112 - DNA, 20 bp, Artificial Sequence
  primer
tgccagcagccgcggtaata

SEQ ID NO: 113 - DNA, 19 bp, Artificial Sequence
  primer
tgccagcagccgcggtaat

SEQ ID NO: 114 - DNA, 18 bp, Artificial Sequence
  primer
tgccagcagccgcggtaa

SEQ ID NO: 115 - DNA, 17 bp, Artificial Sequence
  primer
tgccagcagccgcggta

SEQ ID NO: 116 - DNA, 16 bp, Artificial Sequence
  primer
tgccagcagccgcggt

SEQ ID NO: 117 - DNA, 15 bp, Artificial Sequence
  primer
tgccagcagccgcgg

SEQ ID NO: 118 - DNA, 15 bp, Artificial Sequence
  primer
ttgcgggacttaacc

SEQ ID NO: 119 - DNA, 16 bp, Artificial Sequence
  primer
gttgcgggacttaacc

SEQ ID NO: 120 - DNA, 15 bp, Artificial Sequence
  primer
gttgcgggacttaac

SEQ ID NO: 121 - DNA, 15 bp, Artificial Sequence
  primer
cgttgcgggacttaa

SEQ ID NO: 122 - DNA, 16 bp, Artificial Sequence
  primer
cgttgcgggacttaac

SEQ ID NO: 123 - DNA, 17 bp, Artificial Sequence
  primer
cgttgcgggacttaacc

SEQ ID NO: 124 - DNA, 18 bp, Artificial Sequence
  primer
tcgttgcgggacttaacc

SEQ ID NO: 125 - DNA, 17 bp, Artificial Sequence
  primer
tcgttgcgggacttaac

SEQ ID NO: 126 - DNA, 16 bp, Artificial Sequence
  primer
tcgttgcgggacttaa

SEQ ID NO: 127 - DNA, 15 bp, Artificial Sequence
  primer
tcgttgcgggactta

SEQ ID NO: 128 - DNA, 15 bp, Artificial Sequence
  primer
ctcgttgcgggactt

SEQ ID NO: 129 - DNA, 16 bp, Artificial Sequence
  primer
ctcgttgcgggactta

SEQ ID NO: 130 - DNA, 17 bp, Artificial Sequence
  primer
ctcgttgcgggacttaa

SEQ ID NO: 131 - DNA, 18 bp, Artificial Sequence
  primer
ctcgttgcgggacttaac

SEQ ID NO: 132 - DNA, 19 bp, Artificial Sequence
  primer
ctcgttgcgggacttaacc

SEQ ID NO: 133 - DNA, 20 bp, Artificial Sequence
  primer
gctcgttgcgggacttaacc

SEQ ID NO: 134 - DNA, 19 bp, Artificial Sequence
  primer
gctcgttgcgggacttaac

SEQ ID NO: 135 - DNA, 18 bp, Artificial Sequence
  primer
gctcgttgcgggacttaa

SEQ ID NO: 136 - DNA, 17 bp, Artificial Sequence
  primer
gctcgttgcgggactta

SEQ ID NO: 137 - DNA, 16 bp, Artificial Sequence
  primer
gctcgttgcgggactt

SEQ ID NO: 138 - DNA, 15 bp, Artificial Sequence
  primer
gctcgttgcgggact

SEQ ID NO: 139 - DNA, 15 bp, Artificial Sequence
  primer
cgctcgttgcgggac

SEQ ID NO: 140 - DNA, 16 bp, Artificial Sequence
  primer
cgctcgttgcgggact

SEQ ID NO: 141 - DNA, 17 bp, Artificial Sequence
  primer
cgctcgttgcgggactt

SEQ ID NO: 142 - DNA, 18 bp, Artificial Sequence
  primer
cgctcgttgcgggactta

SEQ ID NO: 143 - DNA, 19 bp, Artificial Sequence
  primer
cgctcgttgcgggacttaa

SEQ ID NO: 144 - DNA, 20 bp, Artificial Sequence
  primer
cgctcgttgcgggacttaac

SEQ ID NO: 145 - DNA, 21 bp, Artificial Sequence
  primer
cgctcgttgcgggacttaacc

(19) United States
(12) Patent Application Publication
(10) Pub. No.: US 2005/0130168 A1
(43) Pub. Date: Jun. 16, 2005

(54) Method of determining a bacterium species

(76) Inventors: Xiang-Yang Han, (US); Audrey S. Pham, (US)

Correspondence Address: Audrey S. Pham, 1211 Andrews, Houston, TX, 77019, US

(21) Appl. No.: 10/697,802

(22) Filed: Oct. 31, 2003

(51) Int. Cl. C12Q 1/68; C12P 19/34

(52) U.S. Cl. 435/6; 435/91.2

(57) ABSTRACT

The present invention provides a rapid and precise test for objectively determining a bacterium species. The assay is applicable to sample isolated from blood, serum, other bodily fluids or environmental samples. The detection method comprises the steps of annealing a region in a DNA template to a specific oligonucleotide primer set comprising SEQ-FOR and SEQ-REV in a complimentary fashion, said primer set designed to give rise to a PCR product having a predetermined size dictated by a complimentary primer set; amplifying said region of DNA template by performing a polymerase chain reaction to produce said PCR product; determining a nucleatide sequence of said PCR product; and comparing said PCR product to a DNA sequence in a database to determine a species of bacterium.

### FIELD OF THE INVENTION

This invention relates generally to determining a species of a bacterium. More particularly, the invention relates to comparing a first nucleotide sequence and a second nucleotide sequence to determine a bacterium species.

### BACKGROUND OF THE INVENTION

Infections by acid-fast organisms pose a major health problem worldwide. For example, mycobacteriosis is caused a Mycobacterium, a slow-growing acid-fast pathogen, and may lead to diseases such as tuberculosis and leprosy. Patients whose immune systems are compromised due to AIDS or side effects of cancer treatments often encounter these infectious agents. Once a host is infected with an acid-fast organism, a series complex of interactions, which involves the organism's ability to survive within the host phagocytic cells and interactions between the host and the organism, results in damaged host tissues. The series of complex interactions makes infections by acid-fast organisms difficult to diagnosis. Often, the organism is determined to be of an acid-fast organism after the host has succumbed to the infection. One long-standing problem is a lack of means to obtain a rapid and accurate diagnosis of infections by acid-fast organisms. Another long-standing problem is a lack of means to provide a determination of the acid-fast organism at early stages of the infection. Often practitioners make up for these deficiencies by administering broad-based treatments as soon as a suspicion of the infection caused by acid-fast organisms arose. Such types of treatment may be time-consuming, may be expensive, or may involve invasive procedures. Several methods have been developed in attempt to resolve these deficiencies. For example, U.S. Pat. No. 6,506,384 issued Jan. 14, 2003 to Laal, et al., discloses an early detection method based on immunoassays; U.S. Pat. No. 5,330,754 issued Jul. 19, 1994 to Kapoor et al., discloses a detection method based on identifying immunodeterminant protein antigens of Mycobacteria. Also, it is well-known in the art that the efficacy of a treatment is directly correlated with at which the stage of infection and the identity of a culprit can be determined. Treatment options and successes are higher if a determination may be made at earlier stages of the infection. Because organisms respond differentially to certain types of drugs, treatments may be better tailored once the organism causing the infection can be determined. Therefore, it is desirable to obtain a rapid and accurate determination of acid-fast organisms.

There is a high demand for a method of achieving an early detection of an acid-fast organism. Methods that relied on detecting bacterial growth in a culture lack an early detection advantage due to the slow growth of the organism. Typically, a nonacid-fast organism will show growth in a culture medium within 24 through 48 hours of incubation at 37 degrees Celsius. A rapid grower that is an acid-fast organism, such as M. fortuitum, may grow within one to two weeks in a nutrient-enhanced medium. For most acid-fast organisms, six through eight weeks of incubation are required to rule out a negative growth (Alcaide et al., 2000, Williams-Bouyer et al., 2000, Tortoli et al., 2001). Following a positive culture, accurate determination of a species of an organism is crucial for proper disease management and infection control. A traditional method of acid-fast staining to identify an acid-fast organism does not provide a determination of specific species. Nucleic acid hybridization (NAH), may yield a rapid identification, but the method is limited to detecting M. tuberculosis complex, M. gordonae, M. kansasii, and M. avium-intracellulare complex. It is infeasible to make probes for the whole hosts of approximately greater than 100 acid-fast species (Hale, et al., 2001; Turenne, et al., 2001). Because probes required for hybridization are available only for these limited number of species, NAH method identified between 58% and 85% of the clinical samples (Williams-Bouyer, et al., 2000; Tortoli, et al., 2001). The remaining 15% through 42% probe-negative isolates have to be identified by other methods. Biochemical tests or mycolic acid analysis have provided alternative methods or have been used in conjunction with NAH method. The requirement of this two-step identification process further delays detection by approximately eight additional weeks. Other two-step identification methods include a gene probing followed by polymerase amplification or radiometric culture combined with gene probe. Such long delays complicate treatment options and increase risks of exposing patients to toxic side effects from potentially unnecessary drug treatment. To further complicate the diagnostic to treatment scheme, the inaccuracy rate of any one particular method, such as biochemical test, mycolic acid analysis or NAH, is approximately 13% to 16% (Patel, et al, 2000; Cloud, et al., 2002).

In recent years, polymerase chain reaction has emerged as a superior method to identify bacteria rapidly (Kolbert, et al., 1999; Drancourt, et al., 2000). Its application to a determination of an acid-fast organism may be particularly useful. Nucleotide acid sequences of 16S ribosomal RNA for most species of acid-fast organism have been identified and submitted to databases, such as the National Institute of Health's GenBank. Yet, such availability of the plethora of nucleotide information has yet to result in a realization of using an amplification method to rapidly and accurately diagnosis of infections by acid-fast organisms. There is a long-felt need to fulfill such gap. The present invention is disclosed and claimed herein provides distinct and useful advantages not previously known to the prior art.

### SUMMARY OF THE INVENTION

The invention is a method for rapid and accurate diagnosis of acid-fast organisms. The advantages of the invention may include one or more of the following.

It is an object of the invention to provide a rapid and accurate method of diagnosis of acid-fast organisms.

It is another object of the invention to provide a new and improved method of diagnosis of bacteria that is simple to use.

It is a further object of the invention to provide an early diagnosis of acid-fast organisms that allows a user to determine appropriate treatments.

These, together with other objects of the invention, along with the various features of novelty that characterize the invention, are pointed out with particularity in the claims annexed to and forming a part of this disclosure. For a better understanding of the invention, its operating and advantages and the specific objects attained by its uses, reference should be had to the accompanying drawings and descriptive matter in which there is illustrated preferred embodiments of the invention.

In some embodiments, the invention relates to a method for determining a bacterium species, comprising annealing a region of a nucleotide template to a specific oligonucleotide primer set comprising SEQ-FOR and SEQ-REV in a complimentary fashion, said primer set designed to provide a product having a predetermined size dictated by a complimentary primer set; amplifying said region of said nucleotide template to produce said product; determining a first nucleotide sequence of said product; and comparing said first nucleotide sequence and a second nucleotide sequence to determine a bacterium species.

In another embodiment, the invention relates to a method for determining a bacterium species, comprising extracting a genomic nucleotide from a specimen to provide a nucleotide template; annealing a region in the nucleotide template to specific oligonucleotide primer set comprising SEQ-FOR and SEQ-REV in a complimentary fashion, said primer set designed to provide to a product having a particular size dictated by the complimentary primer set; amplifying said region of said nucleotide template to produce said product; and determining a species of a bacterium in a nucleotide sequence of said product.

In another embodiment, the invention relates to a method for determining a bacterium species, comprising culturing a bacterium from a specimen; extracting genomic nucleotide from said bacterium to provide a nucleotide template; annealing a desired region in the nucleotide template to a specific oligonucleotide primer set comprising SEQ-FOR and SEQ-REV in a complimentary fashion, said primer set designed to provide a product having a particular size dictated by the complimentary primer set; amplifying said region of said nucleotide template to produce said product; detecting said product; and determining a species of a bacterium from a nucleotide sequence of said product.

In another aspect, the invention relates to a method of determining bacterium species, comprising providing a specimen having a template; providing a pair of primers selected from a group consisting of: (a) a first forward primer having consecutive bases of an AFB-f selected from SEQ ID NO: 18 through SEQ ID NO: 53 or a fragment or a variation thereof and a first reverse primer having consecutive bases of an AFB-r selected from SEQ ID NO: 54 through SEQ ID NO: 89 or a fragment or a variation thereof; (b) a second forward primer having consecutive bases of an UB-f selected from SEQ ID NO: 90 through SEQ ID NO: 117 or a fragment or a variation thereof and a second reverse primer having consecutive bases of an UB-r selected from SEQ ID NO: 118 through SEQ ID NO: 145 or a fragment or a variation thereof; and (c) a first forward primer having consecutive bases of an AFB-f selected from SEQ ID NO: 18 through SEQ ID NO: 53 or a fragment or a variation thereof and a second reverse primer having consecutive bases of an UB-r selected from SEQ ID NO: 118 through SEQ ID NO: 145 or a fragment or a variation thereof; amplifying a region of said template using said pair of primers to produce a product from said specimen; and comparing said product from said specimen with a nucleotide sequence from a database to determine said bacterium species.

In yet another aspect, the invention relates to a method of determining a bacterium species, comprising providing a sample having a template; providing a forward primer having consecutive bases of an AFB-f selected from SEQ ID NO: 18 through SEQ ID NO: 53 or a fragment or a variation thereof and a reverse primer having consecutive bases of an AFB-r selected from SEQ ID NO: 54 through SEQ ID NO: 89 or a fragment or a variation thereof; and amplifying a region of said template using said pair of primers, thereby producing a product from said specimen; and comparing said product from said specimen with a nucleotide sequence from a database to determine said bacterium species present in the specimen.

In another aspect, the invention relates to a method of determining a bacterium species, comprising providing a sample having a template; providing a forward primer having consecutive bases of an UB-f selected from SEQ ID NO: 90 through SEQ ID NO: 117 or a fragment or a variation thereof and a reverse primer having consecutive bases of an UB-r selected from SEQ ID NO: 118 through SEQ ID NO: 145 or a fragment or a variation thereof; amplifying a region of said template using said forward primer or said reverse primer to produce a product from said specimen; and comparing said a product from said specimen with a nucleotide sequence from databases to determine said bacterium species present in the specimen.

### BRIEF DESCRIPTION OF THE DRAWINGS

FIGS. 1A-1L. Comparison of DNA sequences among species of Mycobacteria commonly found in a clinical setting. The sequences are shown in order from 5-prime to 3-prime. The species names are arranged in alphabetical order merely for convenience. The number at the end of the first sequence of each figure represents the nucleotide position for M. heckeshornense.

In FIGS. 1A and 1L, the underlined nucleotides represent a segment of a DNA sequence that is complementary to a primer. FIG. 1A shows the underlined nucleotides representing a segment of a DNA sequence that is complementary to the AFB-f primer (SEQ ID NO: 42). Sequences are aligned according to a match at the 3-prime end of the AFB-f primer, designated as nucleotide “c”. FIG. 1L shows the underlined nucleotides representing a segment of a DNA sequence that is complementary to the AFB-r primer (SEQ ID NO: 82).

FIGS. 1B through 1L represent continuations of the alignments. A vertical bar may be shown between nucleotides of sequences. The vertical bar represents a mismatch between the nucleotides.

FIGS. 2A-2L. Comparison of DNA sequences among species of Mycobacteria commonly found in a clinical setting. The sequences are shown in order from 5-prime to 3-prime. The organisms are arranged by species name in alphabetical order merely for convenience. The number at the end of the first sequence of each figure represents the nucleotide position for M. heckeshornense.

In FIGS. 2A and 2L, the underlined nucleotides represent a portion of a DNA sequence that is complementary to a primer. FIG. 2A shows the underlined nucleotides representing a portion of a DNA sequence that is complementary to the AFB-f primer (SEQ ID NO: 42). Sequences are aligned according to the matches at the 3-prime end of the AFB-f primer, designated as nucleotide “c”. FIG. 2L shows the underlined nucleotides representing a portion of a DNA sequence that is complementary to the AFB-r primer (SEQ ID NO: 82).

FIGS. 2B through 2L represent continuations of the alignments. A vertical bar may be shown between nucleotides of sequences. The vertical bar represents a mismatch between the nucleotides. A dash represents a gap between the nucleotides of a sequence and is used merely for facilitating alignment. For example, in some embodiments a dash may be used between nucleotides to yield the best DNA identity among DNA sequences of species of Mycobacteria.

FIGS. 3A-3B show images of denatured gel electrophoreses showing amplified products of polymerase chain reactions using DNA templates of 16S rRNA from Staphylococcus epidermidis (lane 2), Streptococcus pneumoniae (lane 3), Pseudomonas aeruginosa (lane 4), Neisseria lactamica (lane 5), Branhamella catarrhalis (lane 6), Escherichia coli (lane 7) and Mycobacterium tuberculosis (lane 9). Lane 1 shows a molecular weight ladder from 100 (bottom of ladder) to 1200 (top of ladder) base pairs and lane 8 shows a negative DNA template control.

FIG. 3A shows products of polymerase chain reactions using primers AFB-f (SEQ ID NO: 42) and AFB-r (SEQ ID NO: 82).

FIG. 3B shows products of polymerase chain reactions using primers UB-f (SEQ ID NO: 111) and UB-r (SEQ ID NO: 145).

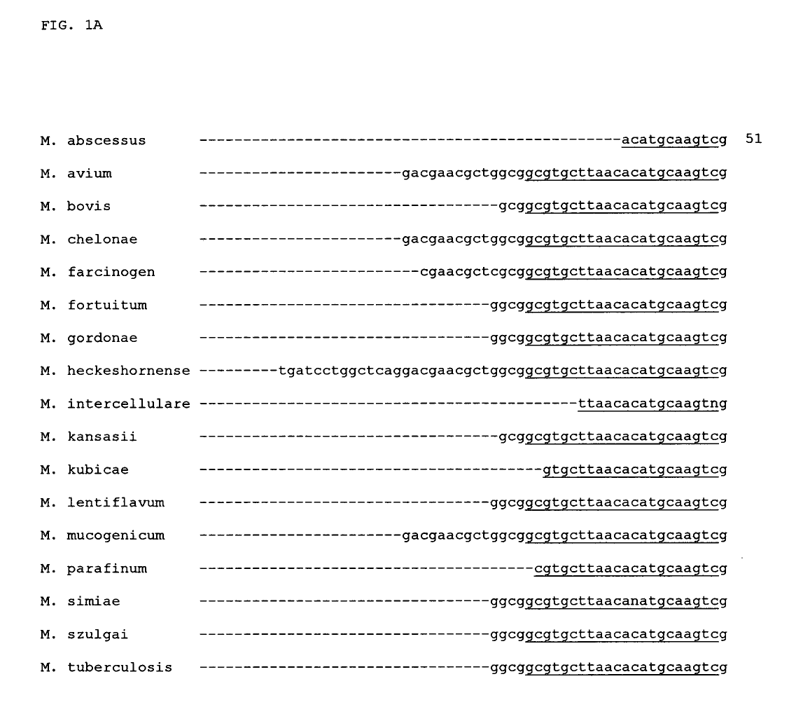

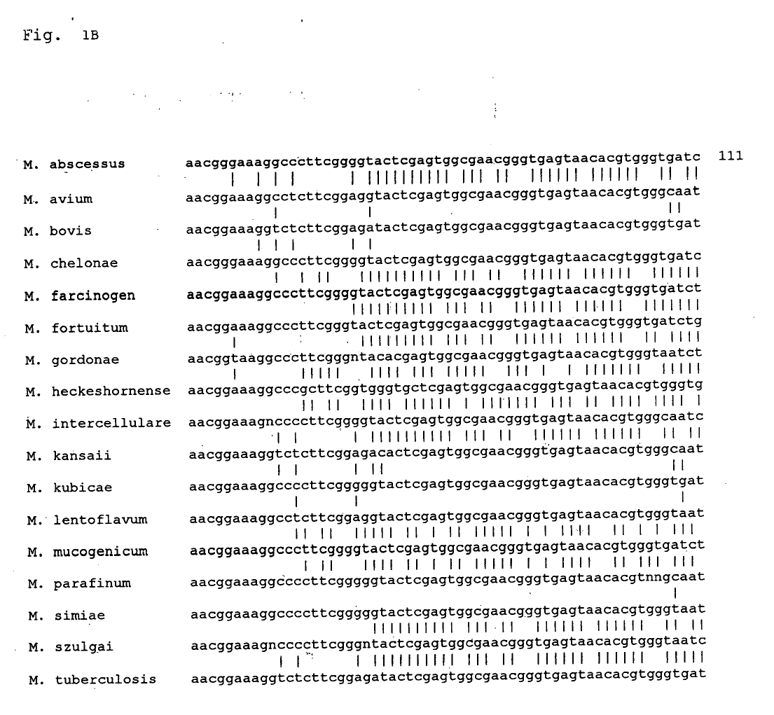

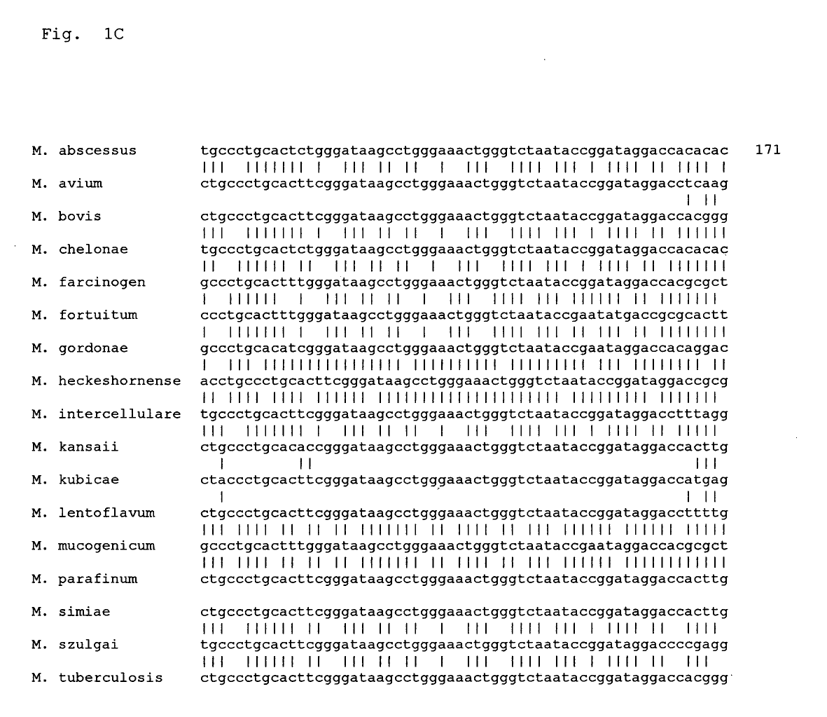

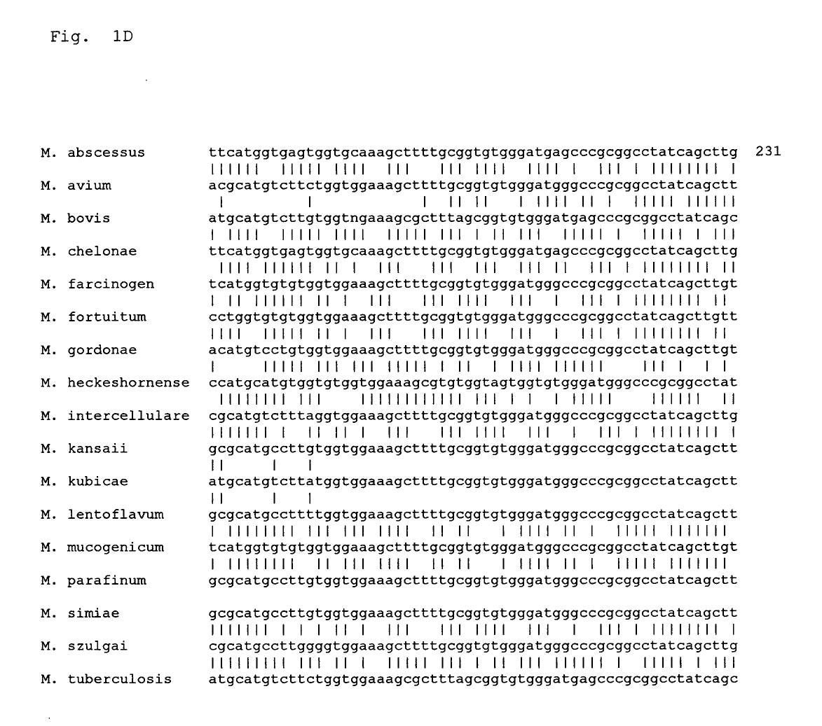

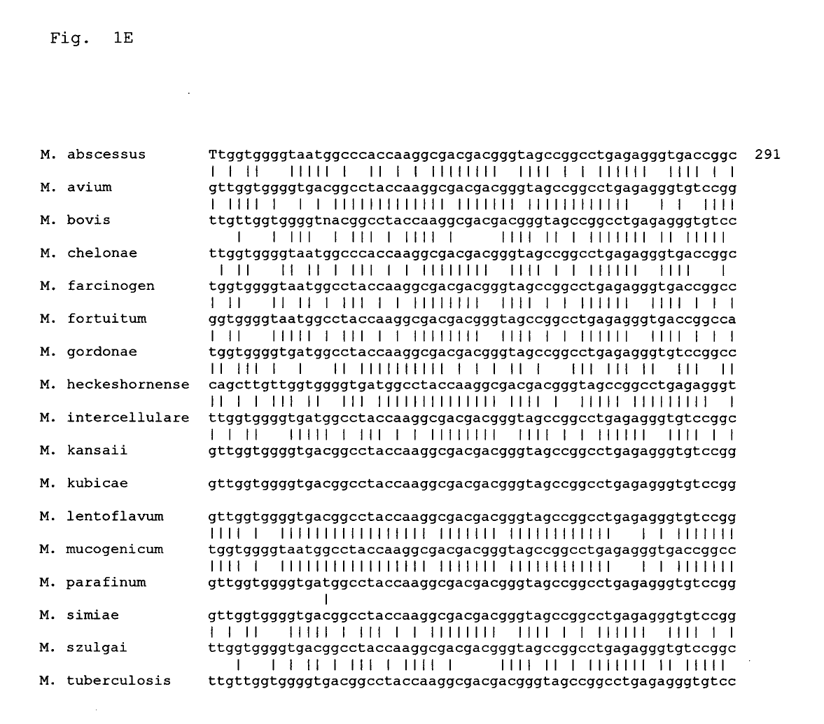

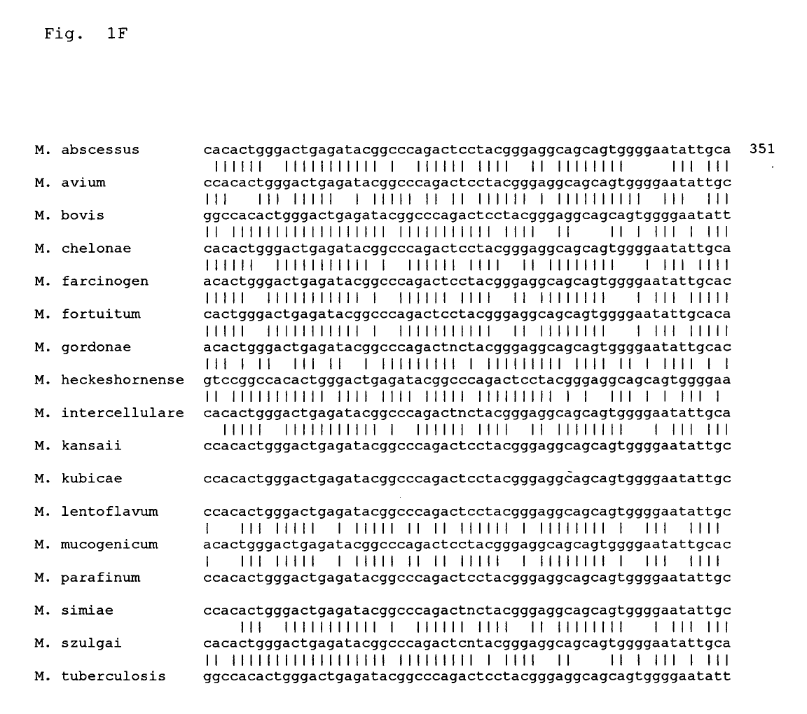

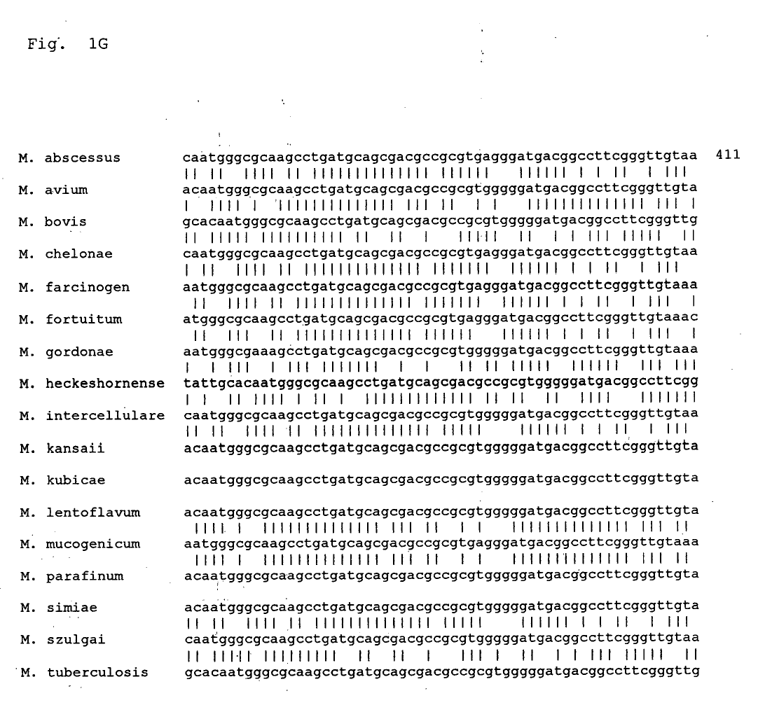

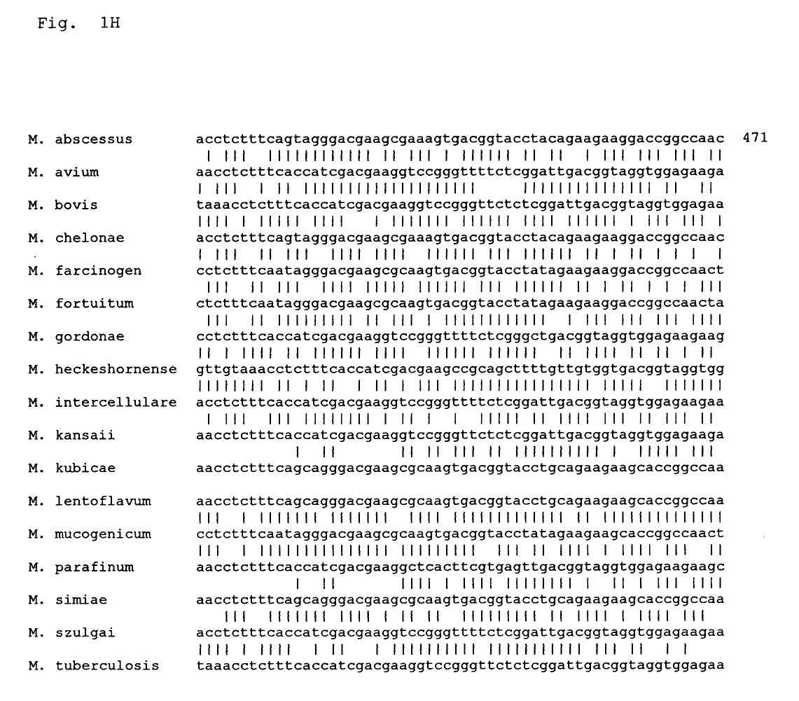

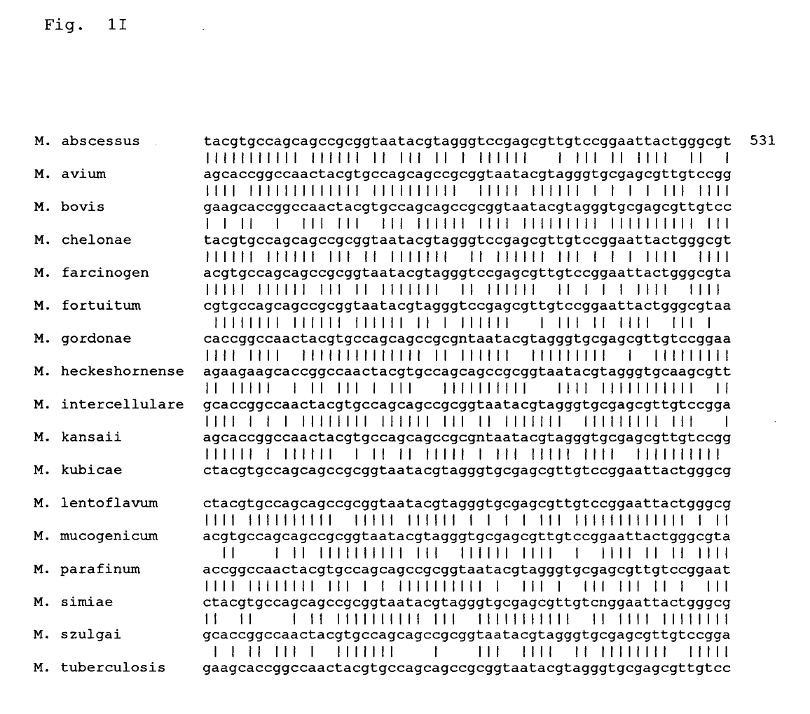

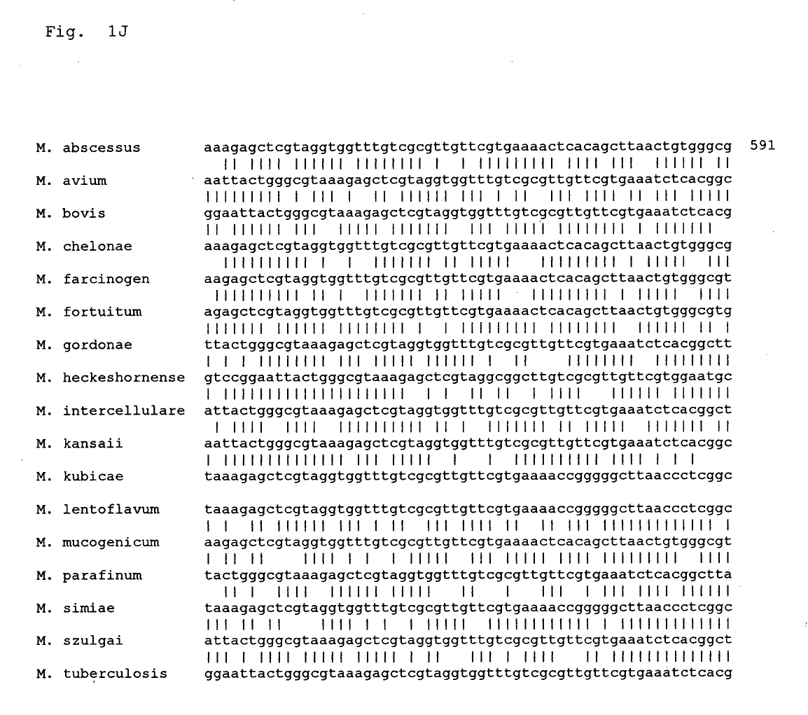

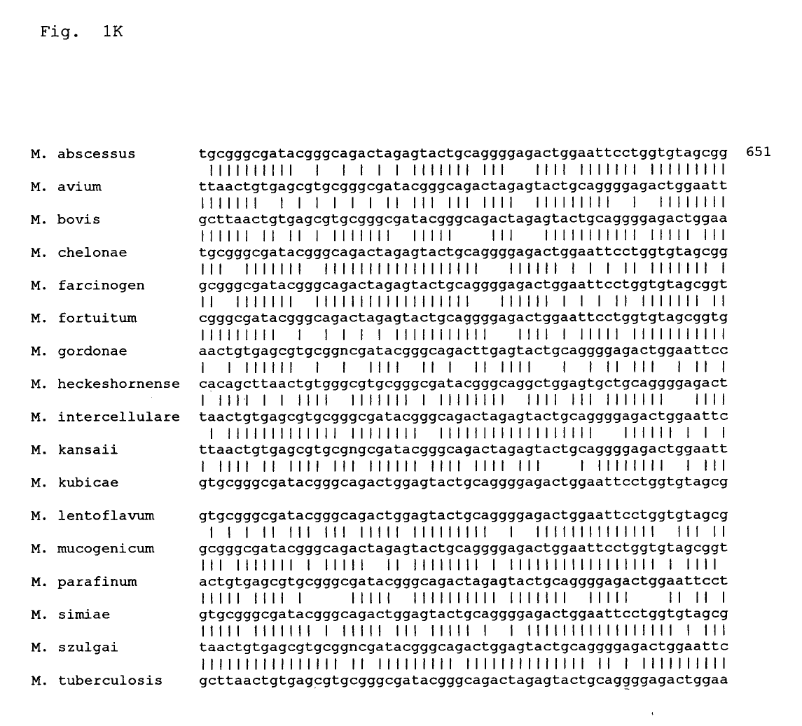

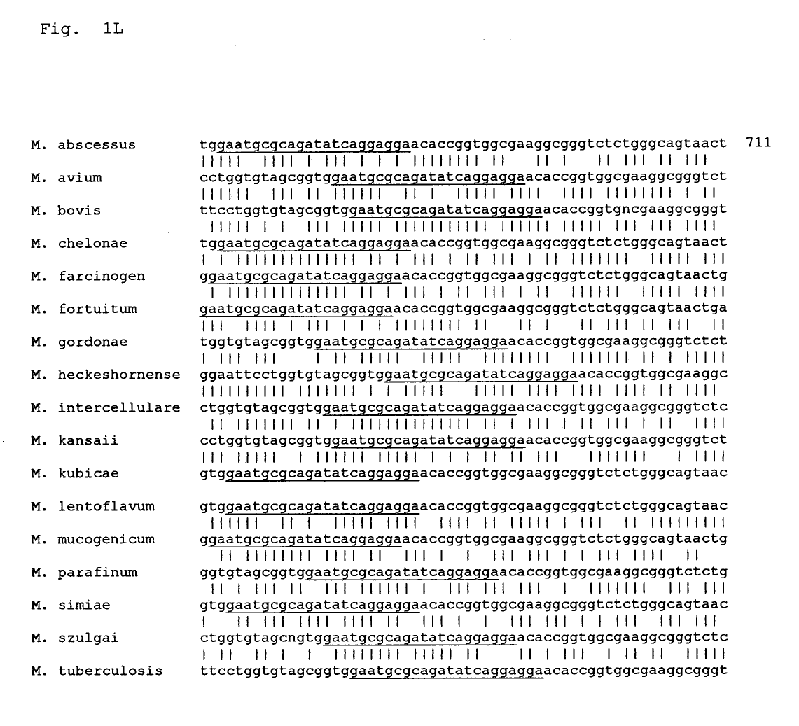

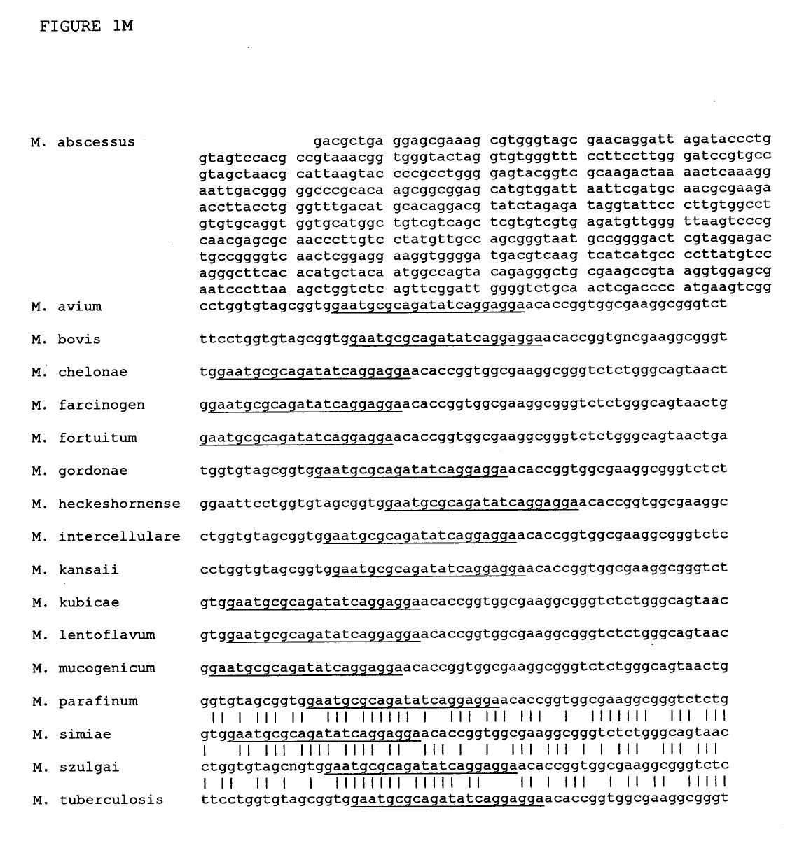

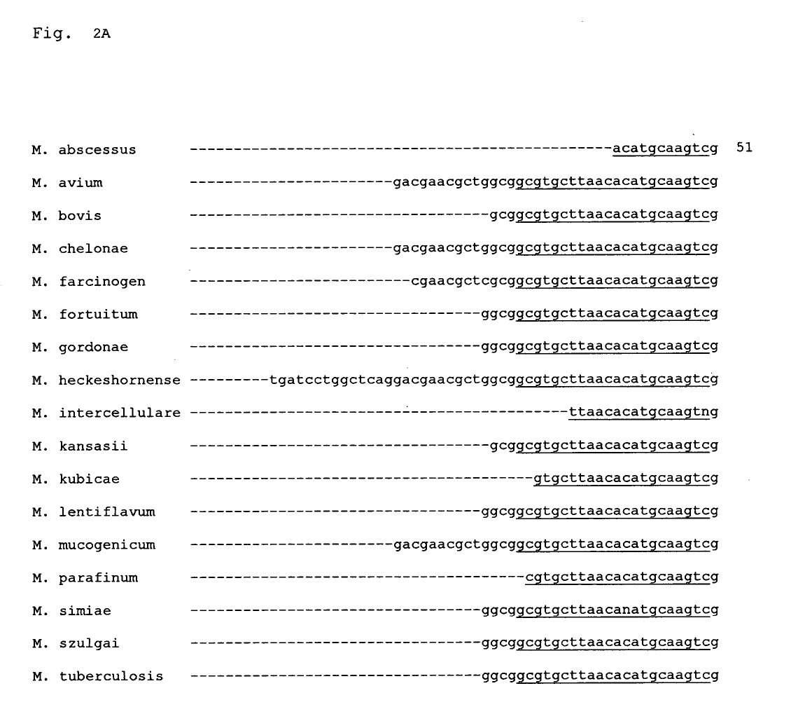

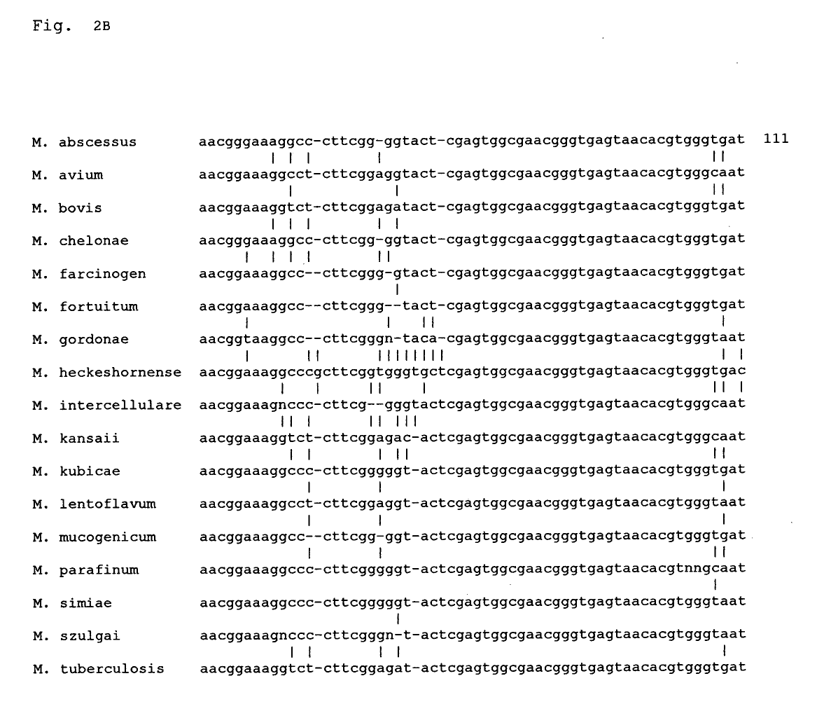

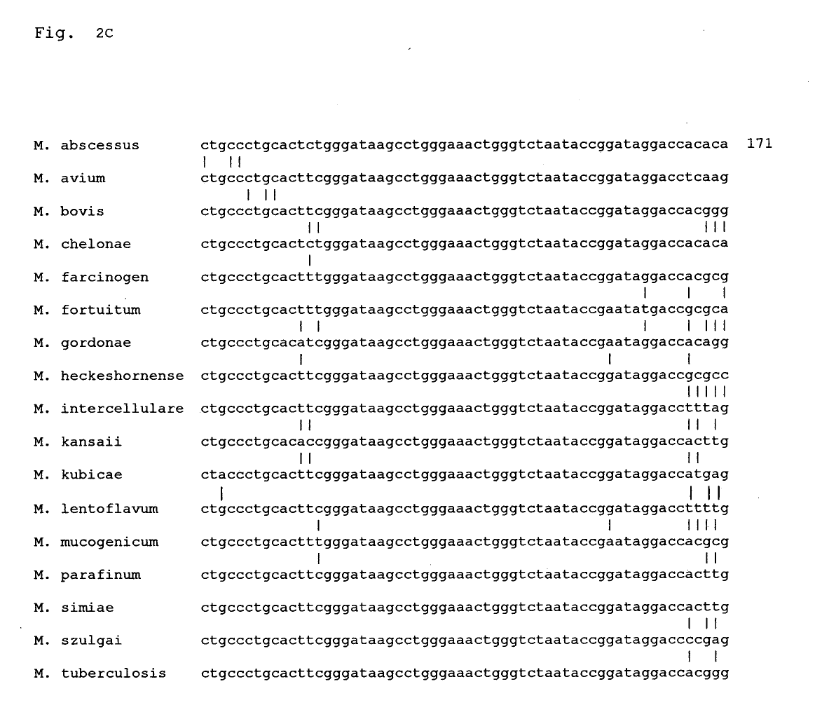

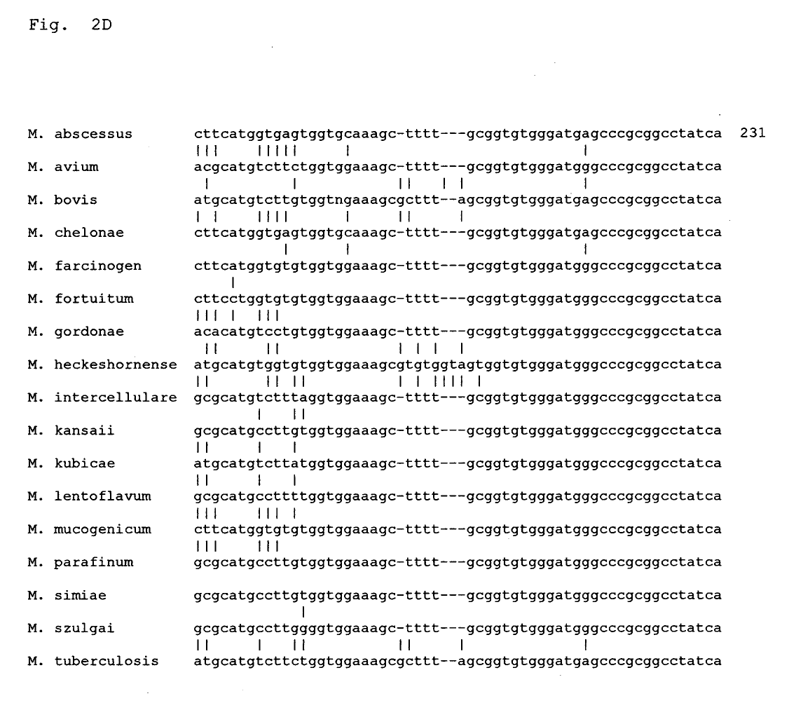

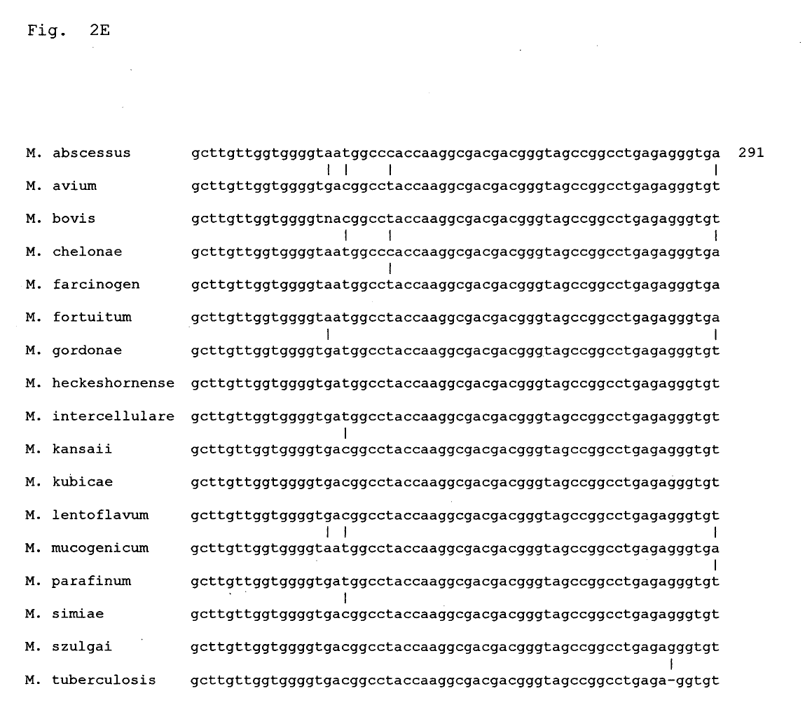

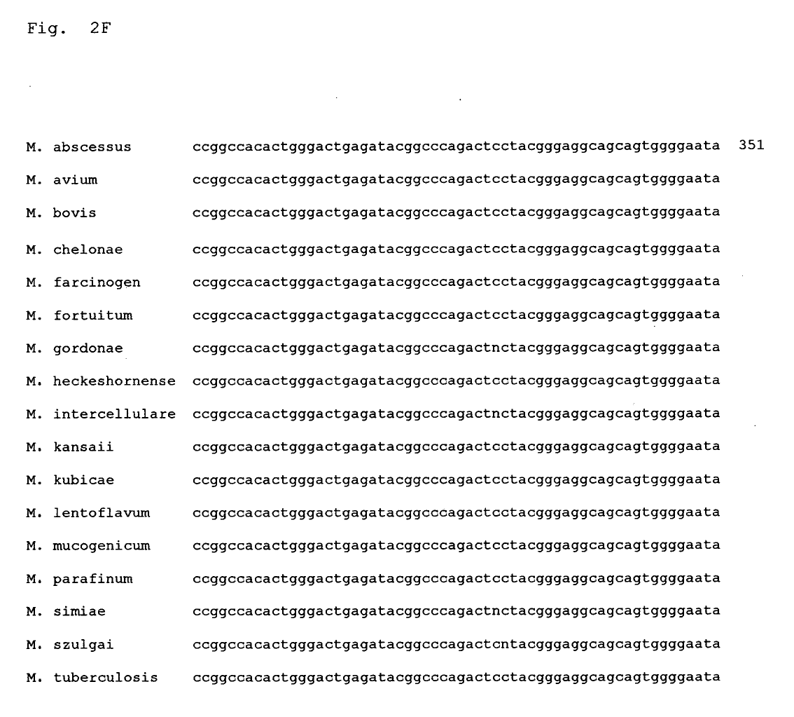

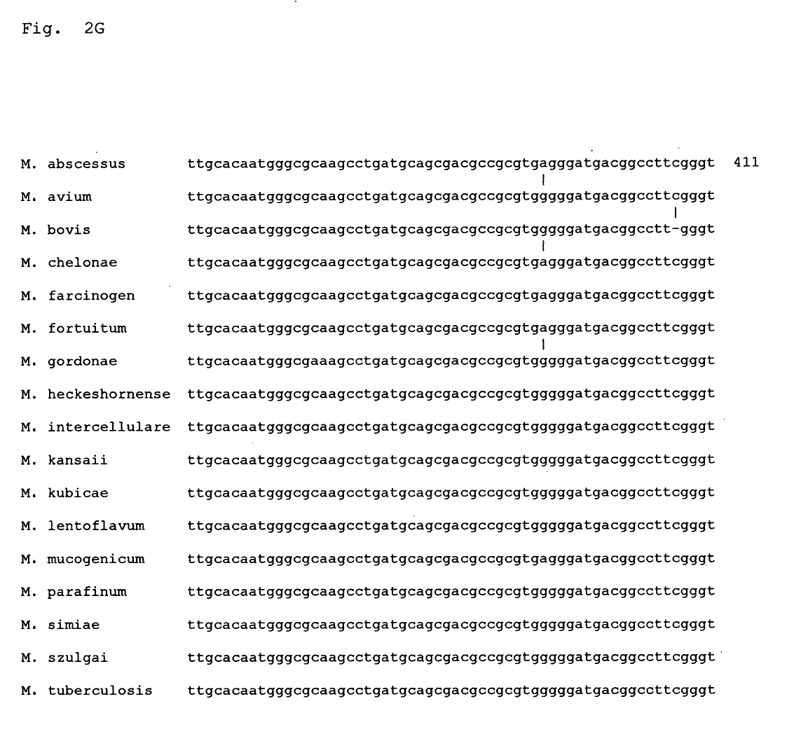

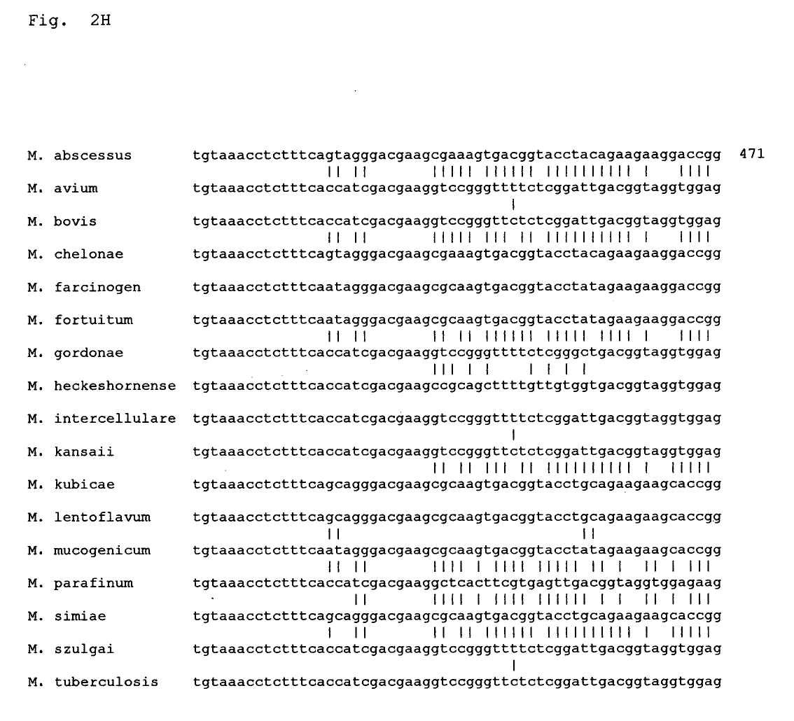

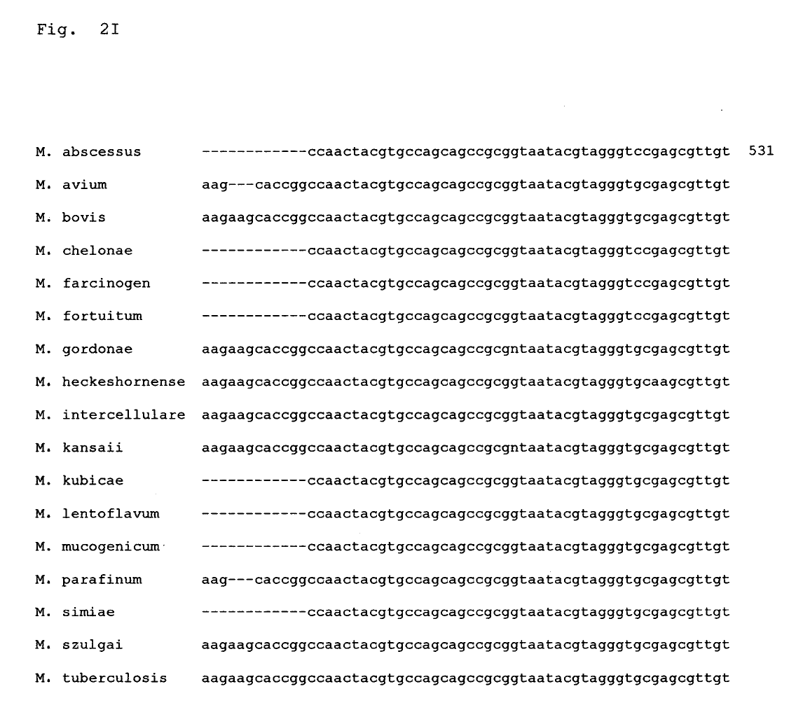

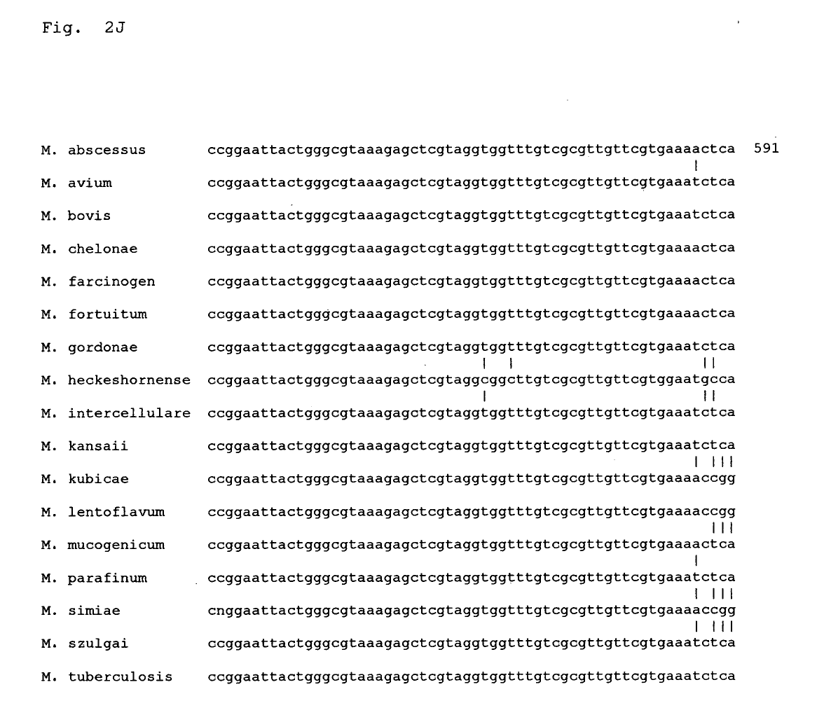

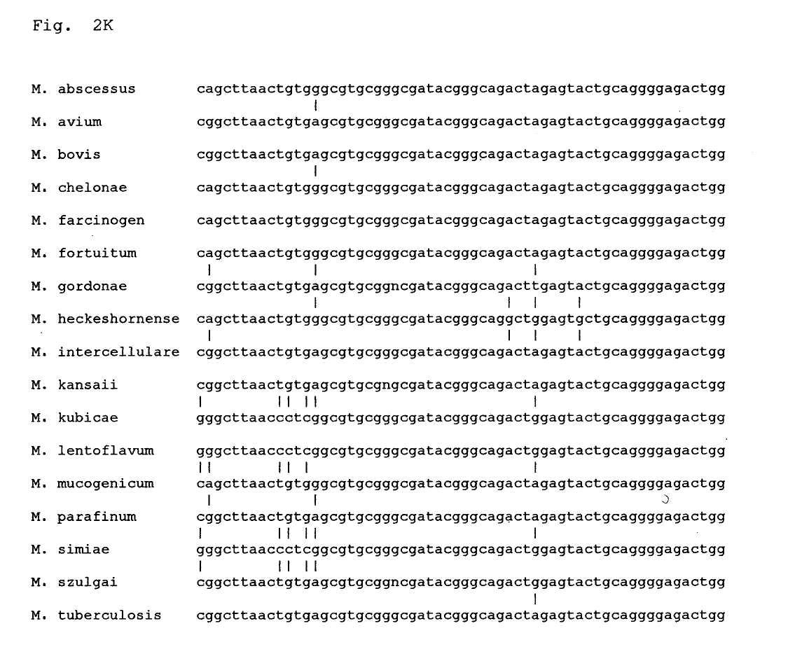

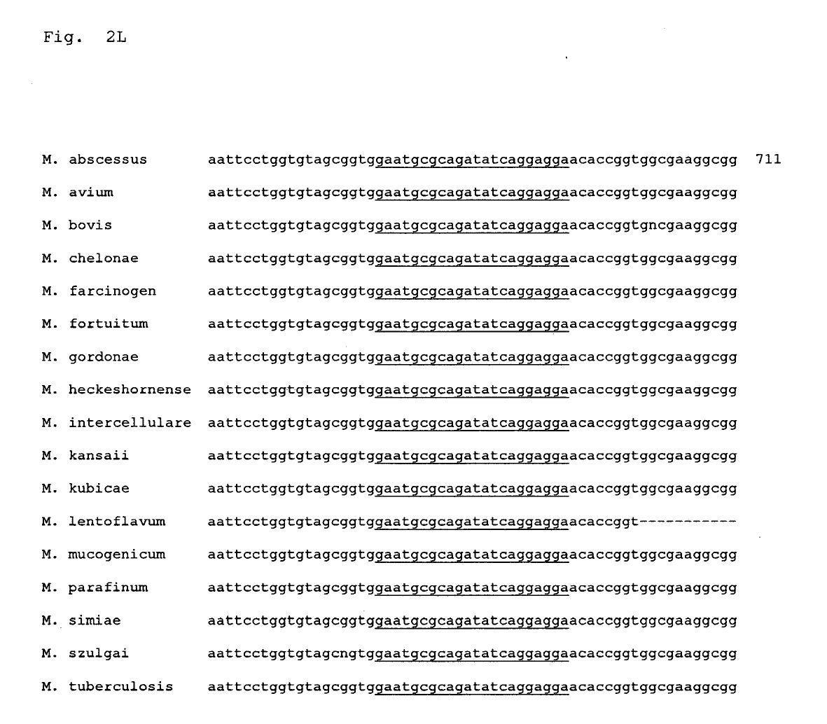

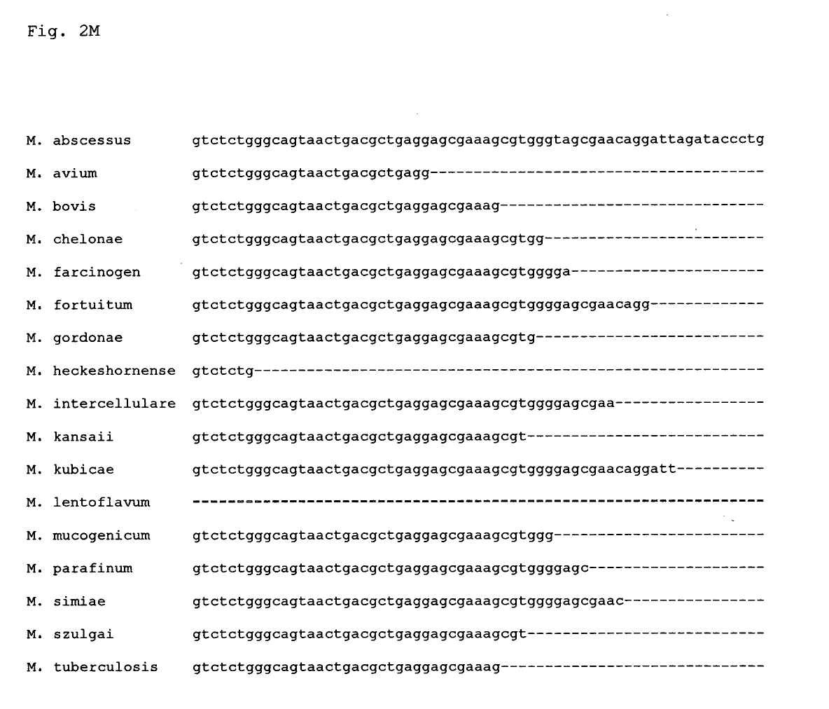

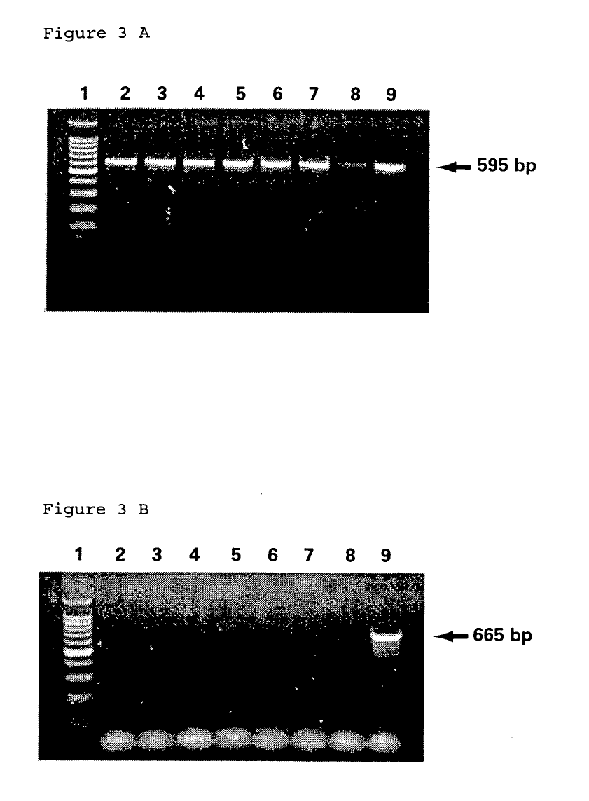

### DETAILED DESCRIPTION OF SOME ILLUSTRATIVE EMBODIMENTS

1. Definitions

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

In the following description, reference will be made to various methodologies known to those of ordinary skill in the art of molecular biology. Commonly used abbreviations in throughout the specification are: AFB, acid-fact bacillus, M., mycobacterium; MAI, mycobacterium avium-intracellulare; MCF, Mycobacterium chelonae-fortuitum; NAH, nucleic acid hybridization; PCR, polymerase chain reaction; DNA, deoxyribonucleotide acid; rDNA, ribosomal DNA; RNA ribonucleotide acid.

As used herein the term “genus” is used to refer to a principal rank in the taxonomic hierarchy, falling below the family level and above the species level. “Species” is used to refer to a fundamental rank in the taxonomic hierarchy falling below the genus level and indicating the limit of organisms able to interbreed; and “strain” is used to refer to a taxonomic level below the species level, which may indicate population variation within a species. Accordingly, PCRs referred to as “species-specific” are those that are adapted to amplify designated regions of DNA from a species. Those PCRs referred to as “genus-specific” are adapted to amplify designated regions of DNA from a number of species within a particular genus.

In some embodiments, the term “hypervariable region” is used to refer to regions within a DNA containing 6-8 base pairs that cyclically repeat along the same stretch. The number of repeats of the hypervariable region is highly polymorphic and almost unique to particular species, strains or individuals. In other embodiments, hypervariable region also refers to a relative lack of DNA sequence similarity within a stretch of DNA among sequences. The term “relative lack of DNA sequence similarity” may mean there is a sufficient distinction in a nucleotide sequence as to distinguish such sequence from other nucleotide sequences or such term may mean the arrangement of the nucleotide sequence is sufficiently distinct as to attribute such distinction of such nucleotide sequence as belonging to a particular species. In some cases, the hypervariable region is relied on to distinguish among species or strains of bacteria or acid-fast bacteria. In other cases, the hypervariable region is not necessary to distinguish among species or strains of bacteria or acid-fast bacteria.

“Oligonucleotide” is used to refer to a segment of DNA that is shorter than the length of the genome of such DNA. According to the invention, the terms segment and fragment may be used interchangeably. An “oligonucleotide primer,” a “nucleotide primer,” a “PCR primer,” and a “primer” are equivalent terms and is used in the instant invention to refer to a nucleotide fragment or an oligonucleotide which is substantially complementary to a nucleotide template sequence and may be adapted to prime the extension of DNA during PCR. According to the invention, a PCR primer is capable of specific hybridization, under appropriate conditions of buffer, ionic strength and temperature, to a region of the DNA template. An oligonucleotide primer may be a DNA fragment of any length sufficient to anneal to a nucleotide template. In some embodiments, an oligonucleotide primer may be a DNA segment of about 10-50 bases long. In other embodiments, an oligonucleotide primer may be a DNA segment of 20-25 bases long. In some embodiments, the oligonucleotide may be designed to hybridize to a hypervariable region of the nucleotide and capable of amplifying multiple copies within a PCR process. In other embodiments, the primer may be designed to anneal to a non-hypervariable region, but is capable of amplifying a hypervariable region. Of course, having a characteristic of hypervariability in a region is not a necessary condition for a primer to hybridize or to amplify copies of a nucleotide sequence.

It is understood that a complementary sequence is one which is capable of forming complement, in which an adenine pairs with a thymine or a guanine pairs with a cytosine. The conditions of PCR is carefully controlled so that the primers will hybridize preferentially to the desired target sequence throughout each amplification cycle, thereby limiting the synthesis of undesirable DNA segment located beyond any PCR primer or outside any primer pair. In an ideal case, each primer would be able to bind only to a desired target in the initial template. In the case of an acid-fast bacterium, it is understood that there will be multiple desired targets in the nucleotide template. Each primer is typically used as a member of a primer pair, including a 5′ upstream primer that hybridizes with the complementary 5′ end of the nucleotide template to be amplified and a 3′ downstream primer that hybridizes with the complementary 3′ end of the nucleotide template to be amplified.

Those of ordinary skill in the art to which the invention relates will understand that the term “complementary” to mean “wholly complementary” or “substantially complementary”, as used herein, means that a primer may have less than 100% complementary to its target template sequence but is still capable of annealing thereto in a specific manner under appropriate conditions.

Generally, “DNA identity” is used to refer to a region of a DNA of a species or organism that is similar or substantially similar to a region of a DNA of another species or organism in the arrangement of nucleotides. In some embodiments, a region that contains a DNA identity among species within a particular genus is referred to as a conserved region at the genus level and is referred to as “genus-specific.” Additionally, in some embodiments, a region that contains a DNA identity among species and among genuses within a particular family is referred to as a conserved region at the family level. In some cases, a region of a DNA may be conserved within a class, order, phylum or kingdom classification. In an embodiment which a primer or primer set is designed from a DNA identity region or regions which are kingdom-specific, the primer or primer set is referred to as “universal primers.” “Primer set,” as used in some instances, is referred to as a pair or multiple primers designed for amplifying a particular region.

According to the invention, “specimen” is used to refer to a sample obtained from a laboratory source; from a central depository including American Type Culture Collection; from a clinical source such as blood, sputum, bronchial alveolar lavage, bronchial wash, tissue biopsy (e.g., lymph node, lung, endometrial curettings, bone, corneal scrapings), body fluids (e.g., cerebral spinal fluid, pleural, synovial, pericardial fluids, peritoneal), pus, urine, bone marrow aspirate, gastric aspirate/wash, stool, tissue fluid (e.g., from eyebrows, ear lobes), implanted medical device, or prosthetic device; or from an environmental source such as soil, sludge, water or liquids.

A method of identification of a bacterium is by staining bacterial cell walls. An acid-fast stain is one type of staining technique in which a bacterium is subjected to a dye and then washed with an acid based solution. An acid-fast bacterium is used to referred to a bacterium that retains the stain after an acid wash. Generally, an acid-fast bacterium is known for comprising a thick durable cell wall in which the dye must be driven into the cell with heat or detergent and, once the stain is embedded in the cell wall, resisted discoloration of an acid solvent wash. The term “AFB” used herein refers to acid-fast bacterium, and may include any bacterium having a character of being acid-fast. AFB, as used in the invention, may be of a Mycobacterium genus. The AFB may be of a genus other than Mycobacterium. Accordingly, the invention may be applied to AFB that is not a Mycobacterium. More specifically, the invention may be applied to AFB that is Corynebacterium, Propionibacterium, Tsukamurella or Actinomyces, Norcardium, to provide some examples. Although some primers described herein contain the names AFB-f or AFB-r, the names of these primers are not correlated with its functions and are not designed to be limiting to amplifying only acid-fast bacteria.

2. Primer Design and Synthesis

The primers of the present invention may be prepared by any number of conventional DNA synthesis methods. In the present case, the primers were manufactured and purchased commercially from Fisher Genosys.

In accordance with some embodiments, optimal results have been obtained using primers which are identical in length and sequence to the primers AFB-f and AFB-r. However, a person of ordinary skill in the art will recognize that a fragment or a variation of a primer may be made to which still maintain the genus-specificity of the PCR amplification and the efficacy of the present inventive method. In some cases, the present invention contemplates that shorter primers containing at least approximately 5 consecutive bases of the nucleotide sequences of these primers may be suitable. Similarly, a primer may be shortened or lengthened. The exact upper or lower limit of the length of the primer is not critical. A typical primer, for example, may be approximately between 15 to 30 bases.

The present invention contemplates minor changes or conservative alterations to a sequence of the primer do not substantially alter its ability to anneal to its specific target DNA and prime extension during PCR. For example, any particular nucleotide, or plurality of nucleotides, of a primer may be substituted for alternative nucleotides, which may not allow for Watson-Crick base-pairing at the particular site of alteration on annealing of the primer to the template DNA during PCR, but nonetheless does not substantially affect the ability of the primer to prime extension during PCR. Such alternative primer may be referred to as an “annealing equivalent” or a “variation” of the primers AFB-f and AFB-r and variant thereof, as described herein. Such annealing equivalents may be adapted to anneal under appropriate conditions for such annealing equivalents. For example, the use of a PCR reaction buffer having 2-7 mM MgCl.sub.2 or an annealing temperature of between 45 degree. C. and 65 degree. C. may be appropriate for most annealing equivalents.

In most embodiments, AFB primers may be capable of amplifying a sequence of Mycobacterium, an acid-fast bacterium, or a non-acid fast bacterium. New bacterial species are routinely discovered and the AFB primers are designed to be capable of annealing to a new or unknown species having a region of a DNA segment that is complementary to the primer. Therefore, AFB-f or AFB-r each independently or as a primer set is capable of amplifying any nucleotide sequence containing a segment of DNA that is complementary to the primer or primers. Example of the primers AFB-f (SEQ ID NO: 42) and AFB-r (SEQ ID NO: 82) and fragments of the primers AFB-f (SEQ ID NO: 18-41 and 43-53), AFB-r (SEQ ID NO: 54-81 and 83-89) which may be appropriate in the present invention are shown below. It is appreciated in the art that variations or other annealing equivalents may be suitable for PCR and are not represented herein. For example, an addition of a nucleotide or nucleotides at the 5-prime or 3-prime end of the primer providing additional complementation to the DNA template may be suitable. To provide another example, an alteration of one or more nucleotides for another within a primer may be suitable.

Fragments of AFB-f (5′-3′)

| | |
|---|---|
| TAACACATGCAAGTC | (SEQ ID NO 18) |
| TTAACACATGCAAGTC | (SEQ ID NO 19) |
| CTTAACACATGCAAGTC | (SEQ ID NO 20) |
| GCTTAACACATGCAAGTC | (SEQ ID NO 21) |
| GCTTAACACATGCAAGT | (SEQ ID NO 22) |
| GCTTAACACATGCAAG | (SEQ ID NO 23) |
| GCTTAACACATGCAA | (SEQ ID NO 24) |
| TGCTTAACACATGCA | (SEQ ID NO 25) |
| TGCTTAACACATGCAA | (SEQ ID NO 26) |
| TGCTTAACACATGCAAG | (SEQ ID NO 27) |
| TGCTTAACACATGCAAGT | (SEQ ID NO 28) |
| TGCTTAACACATGCAAGTC | (SEQ ID NO 29) |
| GTGCTTAACACATGCAAGTC | (SEQ ID NO 30) |
| GTGCTTAACACATGCAAGT | (SEQ ID NO 31) |
| GTGCTTAACACATGCAAG | (SEQ ID NO 32) |
| GTGCTTAACACATGCAA | (SEQ ID NO 33) |
| GTGCTTAACACATGCA | (SEQ ID NO 34) |
| CGTGCTTAACACATG | (SEQ ID NO 35) |
| CGTGCTTAACACATGC | (SEQ ID NO 36) |
| CGTGCTTAACACATGCA | (SEQ ID NO 37) |
| CGTGCTTAACACATGCAA | (SEQ ID NO 38) |
| CGTGCTTAACACATGCAAG | (SEQ ID NO 39) |
| CGTGCTTAACACATGCAAGT | (SEQ ID NO 40) |
| CGTGCTTAACACATGCAAGTC | (SEQ ID NO 41) |
| GCGTGCTTAACACATGCAAGTC | (SEQ ID NO 42) |
| GCGTGCTTAACACATGCAAGT | (SEQ ID NO 43) |
| GCGTGCTTAACACATGCAAG | (SEQ ID NO 44) |
| GCGTGCTTAACACATGCAA | (SEQ ID NO 45) |
| GCGTGCTTAACACATGCA | (SEQ ID NO 46) |
| GCGTGCTTAACACATGC | (SEQ ID NO 47) |
| GCGTGCTTAACACATG | (SEQ ID NO 48) |
| GCGTGCTTAACACAT | (SEQ ID NO 49) |
| TTAACACATGCAAGT | (SEQ ID NO 50) |
| CTTAACACATGCAAG | (SEQ ID NO 51) |
| CTTAACACATGCAAGT | (SEQ ID NO 52) |
| GTGCTTAACACATGC | (SEQ ID NO 53) |

Fragments of AFB-r (5′-3′)

| | |
|---|---|
| GATATCTGCGCATTC | (SEQ ID NO 54) |
| TGATATCTGCGCATTC | (SEQ ID NO 55) |
| TGATATCTGCGCATT | (SEQ ID NO 56) |
| CTGATATCTGCGCAT | (SEQ ID NO 57) |
| CTGATATCTGCGCATT | (SEQ ID NO 58) |
| CTGATATCTGCGCATTC | (SEQ ID NO 59) |
| CCTGATATCTGCGCATTC | (SEQ ID NO 60) |
| CCTGATATCTGCGCATT | (SEQ ID NO 61) |
| CCTGATATCTGCGCAT | (SEQ ID NO 62) |
| CCTGATATCTGCGCA | (SEQ ID NO 63) |
| TCCTGATATCTGCGC | (SEQ ID NO 64) |
| TCCTGATATCTGCGCA | (SEQ ID NO 65) |
| TCCTGATATCTGCGCAT | (SEQ ID NO 66) |
| TCCTGATATCTGCGCATT | (SEQ ID NO 67) |
| TCCTGATATCTGCGCATTC | (SEQ ID NO 68) |
| CTCCTGATATCTGCGCATTC | (SEQ ID NO 69) |
| CTCCTGATATCTGCGCATT | (SEQ ID NO 70) |
| CTCCTGATATCTGCGCAT | (SEQ ID NO 71) |
| CTCCTGATATCTGCGCA | (SEQ ID NO 72) |
| CTCCTGATATCTGCGC | (SEQ ID NO 73) |
| CTCCTGATATCTGCG | (SEQ ID NO 74) |
| CCTCCTGATATCTGC | (SEQ ID NO 75) |
| CCTCCTGATATCTGCG | (SEQ ID NO 76) |
| CCTCCTGATATCTGCGC | (SEQ ID NO 77) |
| CCTCCTGATATCTGCGCA | (SEQ ID NO 78) |
| CCTCCTGATATCTGCGCAT | (SEQ ID NO 79) |
| CCTCCTGATATCTGCGCATT | (SEQ ID NO 80) |
| CCTCCTGATATCTGCGCATTC | (SEQ ID NO 81) |
| TCCTCCTGATATCTGCGCATTC | (SEQ ID NO 82) |
| TCCTCCTGATATCTGCGCATT | (SEQ ID NO 83) |
| TCCTCCTGATATCTGCGCAT | (SEQ ID NO 84) |
| TCCTCCTGATATCTGCGCA | (SEQ ID NO 85) |
| TCCTCCTGATATCTGCGC | (SEQ ID NO 86) |
| TCCTCCTGATATCTGCG | (SEQ ID NO 87) |
| TCCTCCTGATATCTGC | (SEQ ID NO 88) |
| TCCTCCTGATATCTG | (SEQ ID NO 89) |

In accordance with some embodiments, each primer presented above may be complementary to 16S rDNA sequences of most Mycobacteria species or most acid-fast bacterium. While the novel primers disclosed herein have been designed to enable genus-specific PCR amplification of regions of 16S rDNA, the region of the amplified DNA sequence, also known as a region contained within the primers, is a hypervariable region. It will be appreciated that species-specific identification of a particular Mycobacterium or acid-fast bacterium may be obtained by comparing the hypervariable region to a known sequence or to DNA sequences in a database.

In some embodiments, Mycobacteria, other acid-fast bacterium, and other bacteria such as Escherichia coli may be amplified using a universal primer pair. Such universal primer pairs are substantially complementary to most species of bacteria and may be used to amplify regions between the primers, whereas such regions may not confer similar sequence identity. Example of the primers UB-f (SEQ ID NO: 111) and UB-r (SEQ ID NO: 145) and fragments of the primers UB-f (SEQ ID NO: 90-110 and 112-117), UB-r (SEQ ID NO: 118-144) which may be appropriate in the present invention are shown below.

Variation of UB-f (5′-3′)

| | |
|---|---|
| CAGCCGCGGTAATAC | (SEQ ID NO 90) |
| GCAGCCGCGGTAATAC | (SEQ ID NO 91) |
| GCAGCCGCGGTAATA | (SEQ ID NO 92) |
| AGCAGCCGCGGTAATA | (SEQ ID NO 93) |
| AGCAGCCGCGGTAATAC | (SEQ ID NO 94) |
| AGCAGCCGCGGTAAT | (SEQ ID NO 95) |
| CAGCAGCCGCGGTAA | (SEQ ID NO 96) |
| CAGCAGCCGCGGTAAT | (SEQ ID NO 97) |
| CAGCAGCCGCGGTAATA | (SEQ ID NO 98) |
| CAGCAGCCGCGGTAATAC | (SEQ ID NO 99) |
| CCAGCAGCCGCGGTAATAC | (SEQ ID NO 100) |
| CCAGCAGCCGCGGTAATA | (SEQ ID NO 101) |
| CCAGCAGCCGCGGTAAT | (SEQ ID NO 102) |
| CCAGCAGCCGCGGTAA | (SEQ ID NO 103) |
| CCAGCAGCCGCGGTA | (SEQ ID NO 104) |
| GCCAGCAGCCGCGGT | (SEQ ID NO 105) |
| GCCAGCAGCCGCGGTA | (SEQ ID NO 106) |
| GCCAGCAGCCGCGGTAA | (SEQ ID NO 107) |
| GCCAGCAGCCGCGGTAAT | (SEQ ID NO 108) |
| GCCAGCAGCCGCGGTAATA | (SEQ ID NO 109) |
| GCCAGCAGCCGCGGTAATAC | (SEQ ID NO 110) |
| TGCCAGCAGCCGCGGTAATAC | (SEQ ID NO 111) |
| TGCCAGCAGCCGCGGTAATA | (SEQ ID NO 112) |
| TGCCAGCAGCCGCGGTAAT | (SEQ ID NO 113) |
| TGCCAGCAGCCGCGGTAA | (SEQ ID NO 114) |
| TGCCAGCAGCCGCGGTA | (SEQ ID NO 115) |
| TGCCAGCAGCCGCGGT | (SEQ ID NO 116) |
| TGCCAGCAGCCGCGG | (SEQ ID NO 117) |

Variation of UB-r (5′-3′)

| | |
|---|---|
| TTGCGGGACTTAACC | (SEQ ID NO 118) |
| GTTGCGGGACTTAACC | (SEQ ID NO 119) |
| GTTGCGGGACTTAAC | (SEQ ID NO 120) |
| CGTTGCGGGACTTAA | (SEQ ID NO 121) |
| CGTTGCGGGACTTAAC | (SEQ ID NO 122) |
| CGTTGCGGGACTTAACC | (SEQ ID NO 123) |
| TCGTTGCGGGACTTAACC | (SEQ ID NO 124) |
| TCGTTGCGGGACTTAAC | (SEQ ID NO 125) |
| TCGTTGCGGGACTTAA | (SEQ ID NO 126) |
| TCGTTGCGGGACTTA | (SEQ ID NO 127) |
| CTCGTTGCGGGACTT | (SEQ ID NO 128) |
| CTCGTTGCGGGACTTA | (SEQ ID NO 129) |
| CTCGTTGCGGGACTTAA | (SEQ ID NO 130) |
| CTCGTTGCGGGACTTAAC | (SEQ ID NO 131) |
| CTCGTTGCGGGACTTAACC | (SEQ ID NO 132) |
| GCTCGTTGCGGGACTTAACC | (SEQ ID NO 133) |
| GCTCGTTGCGGGACTTAAC | (SEQ ID NO 134) |
| GCTCGTTGCGGGACTTAA | (SEQ ID NO 135) |
| GCTCGTTGCGGGACTTA | (SEQ ID NO 136) |
| GCTCGTTGCGGGACTT | (SEQ ID NO 137) |
| GCTCGTTGCGGGACT | (SEQ ID NO 138) |
| CGCTCGTTGCGGGAC | (SEQ ID NO 139) |
| CGCTCGTTGCGGGACT | (SEQ ID NO 140) |
| CGCTCGTTGCGGGACTT | (SEQ ID NO 141) |
| CGCTCGTTGCGGGACTTA | (SEQ ID NO 142) |
| CGCTCGTTGCGGGACTTAA | (SEQ ID NO 143) |
| CGCTCGTTGCGGGACTTAAC | (SEQ ID NO 144) |
| CGCTCGTTGCGGGACTTAACC | (SEQ ID NO 145) |

While the novel primers disclosed herein have been designed to enable PCR amplification of regions of 16S rDNA, it will be appreciated that they may be also applied, individually or in combination, to various other applications. For example, they may be used as molecular probes, or primers for alternative molecular techniques.

Generally, only one PCR, using a single primer set, will be needed in order to identify the species of Mycobacteria present within a sample. However it will be appreciated that there may be time where a parallel PCR, using a second primer set, may be utilized in order to further clarify the identity of a species present within a sample. Similarly, upon optimization of the PCR condition both novel primer sets may be used in a single PCR.

3. DNA Purification

It will be appreciated by those of ordinary skills in the art that methods of purifying nucleotide from the bacteria varied widely as with the total amount of nucleotide product obtained from these methods. Most methods, however, do adhere to some fundamental techniques. Generally, cells are ruptured to expose the DNA and then the nucleotides are separated from the crude mixture. Some methods of rupturing cells may include the use of a detergent such as sodium dodecyl sulfate or a solvent such as phenol-chloroform. Nucleotides may be separated from the crude extract by use of physical methods such as centrifugation, pressure techniques or by introduction of a medium, such as silica, for the nucleotide to bind. After sufficient washing, the pure nucleotides may be isolated and suspended in either water or a buffered solution. Some methods are more labor intensive and time consuming than other methods. For example, a method of nucleotide purification by phenol-chloroform extraction may require repeated extraction of each step for at least two times to ensure a nucleotide yield of high purity. In some embodiments, a method of rapid purification may involve the combined processes of extraction, separation and suspension of DNA in one Prepman™ Ultra solution.

4. PCR Amplification

Each PCR is performed with at least one monospecific control sample or standard of known species identity. It will be appreciated that controls containing more than one known species may be entertained. Positive controls which include, or negative controls which exclude, nucleotide templates of Mycobacteria or acid-fast bacteria may be used. It will be appreciated that other controls routinely used in the art may also be run against the samples. The nucleotide templates of the controls or standards of known species may be obtained from American Tissue Culture Collection and purified accordingly.

Amplification is conducted according to procedures in the art to which this invention relates; such as described in U.S. Pat. No. 4,683,202. According to some embodiments, the amplification reaction mixture may include 50 nano.M -200.mu.M of each primer, 100-200 mu.M each dNTP, 2-7 mM MgCl.sub.2, or 1-2 U Taq DNA polymerase (Promega). In some cases, additional amounts of magnesium or glycerol may be added to enhance an amplification reaction. In some embodiments, PCR cycling may be performed under the following conditions: denaturation at a temperature of 92-95 .degree. C. for 30-60 seconds, annealing at a temperature from 45.degree. C. to 60 degree. C. for 30-60 seconds and extension at a temperature of 65-75 degree. C. for 30-60 seconds. In some embodiments, an initial incubation period of 2-15 minutes at a temperature of 92-95 . degree.C. may be used to activate the enzyme. Also in some embodiments, a final incubation period of approximately 2-5 minutes may be used to facilitate synthesis of any frayed end-fragment. In most cases, between 20 and 40 cycles may be performed. For example, the following PCR conditions may be pairs of primers AFB-b and AFB-r, and UB-f and UB-r, of the invention:

| Initial | | | | |
|---|---|---|---|---|
| Additional | | | | |
| Activation | | | | Cy- |
| Extension | Denaturation | Annealing | Extension | cles |
| — | 95° C., 20 sec | 55° C., 20 sec | 72° C., 20 sec | 35 |
| 72° C., 300 sec | | | | |
| 92° C., 120 sec | 95° C., 20 sec | 55° C., 20 sec | 72° C., 20 sec | 35 |
| 72° C., 300 sec | | | | |
| 92° C., 120 sec | 95° C., 20 sec | 55° C., 20 sec | 72° C., 20 sec | 30 |
| 72° C., 300 sec | | | | |
| 92° C., 120 sec | 95° C., 20 sec | 58° C., 20 sec | 72° C., 20 sec | 30 |
| 72° C., 300 sec | | | | |

It will be appreciated by those of ordinary skill in the art that the PCR conditions provided herein are merely examples and may be varied so as to optimize conditions where, for example, alternative PCR cycler or DNA polymerase are used, where the quality of the template DNA differs, or where variations of the primers may not specifically exemplified herein are used, without departing from the scope of the present invention. The PCR conditions may be altered or optimized by changing the concentration of the various constituents within the reaction, altering the number of amplification cycles, the denaturation, annealing or extension times or temperatures, or the quantity of template DNA, for example. It will be realized that the specificity of the annealing is most important in the first several amplification cycle. Those skill in the art will appreciate there are a number of other ways in which PCR conditions may be optimized to overcome variability between reactions.

It will be understood that where no specificity exemplified herein appropriate PCR annealing temperatures for any primers within the scope of the present invention may be derived from the calculated melting temperature of that primer. Such melting temperature may be calculated using standard formulas, such as that described in Sambrook, 2001. As will be understood by those of ordinary skill in the art to which this invention relates annealing temperature may be above or below the melting temperature but generally an annealing temperature of approximately 0.degree. C to 10.degree. C. below the calculated melting temperature of the primer may be suitable.

5. Determination of PCR Product

Conditions for specific hybridization of primers to particular template targets is determined empirically, by varying the annealing temperature in several degree increments and comparing the specificity and efficiency of the amplification process by a visualization technique such as agarose mini-gel electrophoresis of an aliquot of the product.

PCR products, obtained from the amplification of the hypervariable regions of both unknown samples and relevant control samples or standards of known identities, may be detected by electrophoretic separation. Electrophoretic techniques which are particularly sensitive to minor differences in PCR product size and/or sequence may be preferred. In some cases, subjecting the sample to electrophoresis may be unnecessary.

5. Species-Specific Determination of Nucleotide Sequence

According to the invention, the term BLAST represents an acronym for Basic Local Alignment Search Tool, an algorithm program used to identify an unknown sequence in a database (Altschul, et al., 1990). The sequence may include a sequence derived from nucleotides, proteins, or genomes. Variations of the BLAST program includes MEGABLAST, for aligning longer sequence, nucleotide BLAST, for finding sequences similar, but not identical, to a query, protein BLAST for protein searches, PSI-BLAST for more sensitive protein similarity searches, PHI-BLAST for a restricted protein pattern searches, RSP-BLAST to identify conserved protein domains, and the like. The BLAST nucleotide algorithm finds similar sequence by generating an indexed table or dictionary of short subsequences called words for both a query and a database. The program then finds initial exact matches to the query words by simply looking up a particular word in the database dictionary. These initial matches are starting point for longer alignments that are generated in several steps, ending with a final gapped alignment. “Word size” is roughly referred to as a minimum length of an identical match an alignment must contain if it is to be found by the algorithm. One of the variables that may be adjusted is the length of a word size, which may be reduced from a value of 64 to a minimum of 7 to increase sensitivity. The standard nucleotide BLAST is better as finding sequences similar, but not identical, to a query. The Megablast has a relaxed 99% identity cutoff and may be the tool of choice to identify a sequence. The default wordsize for the Megablast is set at 28. The Megablast is specifically designed to find long alignments between very similar sequences and thus may be the best tool to find identical matches to match a query sequence. The expect value is the statistical significance threshold for reporting matches against sequences in a database. The expect value significance cut-off is set at 10, which indicates that 10 matches are expected to be found merely by chance, according to the stochastic model of Karlin and Altschul (1990). If the statistical significance ascribed to a match is greater than the expected threshold, the match may not be reported. Lower expect thresholds may be more stringent, leading to fewer chance matches being reported. Increasing the threshold may show less stringent matches.

The default databases used in an algorithm analyses includes the non-redundant database of GenBank, in which sequences may be derived from the 3-dimensional structure Brookhaven Protein Data Bank, EMBL Nucleotide Sequence Database from the European Bioinformatics Institute. The algorithm may exclude the EST (expressed sequence tag), STS (sequence tagged site), GSS (genome survey sequence) or HTGS (high throughput genomic sequencing) databases.

The invention will be further clarified by the following example, which is intended to be purely an example of the invention.

### EXAMPLE

Design of Primers

Deoxyribonucleotide sequences of 16S ribosomal RNA are aligned to determine a position, a segment, positions or segments of nucleotide matches and mismatches among species belonging to acid-fast bacteria. Genus-specific oligonucleotide primers are designed from regions of DNA segment containing DNA identity among all species compared. In this example, the genus-specific primers used in the experiment are forward primer 5′-GCGTGCTTAACACATGCAAGTC-3′ and reverse primer 5′-TCCTCCTGATATCTGCGCATTC-3′. These primers are designed to amplify a region containing hypervariable regions A and B (Rogall, et al., 1990; Holberg-Peterson, 1999; Tortoli, et al., 2001). The size of the pre-determined fragment is estimated to be from 550 through 665 base pairs, depending on the species. The universal primers used in this example are: 5′-TGCCAGCAGCCGCGGTAATAC-3′ and 5′-CGCTCGTTGCGGGACTTAACC-3′. The estimated fragment length is between 500 through 700, depending on the organism. In some cases, the genus-specific forward primer 5′-GCGTGCTTAACACATGCAAGTC-3′ may be used with the universal reverse primer 5′-CGCTCGTTGCGGGACTTAACC-3′ to amplify a DNA region of an organism. The size of the pre-determined fragment is estimated to be from 1000 through 1700 base pairs, depending on the organism. None of the primer combinations set forth herein has DNA identity to eukaryotic or viral DNA sequences.

DNA Extraction and PCR

Bacteria are harvested from pelleting 1 mL of a liquid medium or from selecting 2-3 colonies of a solid medium after incubation. The estimated number of cells harvested from the 1 mL-pellet or 2 colonies is approximately 1×10¹⁰ cells. The harvested cells are placed in a sterile receptacle containing 200 mu.L PrepMan™ Ultra extraction solution (Applied Biosystems) and boiled for 10 minutes. Vortexing is performed before and after boiling to ensure sufficient cell breakage. The sample is allowed to come to room temperature after boiling and then spun at 8,000×g for 5 minutes. The supernatant is then transferred to another sterile receptacle.

Typically, 1 through 5 mu.L of the extracted supernatant is used as a template in a 50 mu.L PCR mixture. The reaction mixture may contain a 1 mu.L of each of the primers, 100 mu.L of each of the deoxynucleoside triphosphates, 2 U of YieldAce™ Taq polymerase (Stratagene, La Jolla, Calif.), an appropriate amount of buffer as commonly determined by the type of polymerase, and an appropriate amount of distilled water to bring the mixture volume up to 50 mu.L. In this example, the PCR condition was carried out as follows: 92 degrees. C. for 120 seconds; 35 reaction cycles at 95 degrees. C. for 20 seconds, 55° C. for 20 seconds, and 72° C. for 20 seconds; and then 72° C. for 300 seconds.

In some instances, denatured gel electrophoreses were performed to confirm the presence of PCR products. Typically, 5 mu.L was examined on a 1.5% agarose gel stained with ethidium bromide.

Nucleotide Sequencing and Identification

PCR product was treated with exonuclease and shrimp alkaline phosphatase (US Biochemicals, Cleveland, Ohio) to remove any unpolymerized primer and deoxynucleoside triphosphate. Sequencing was performed by a dye-terminator method in an ABI 377 model sequencer (Applied Biosystems). The result of sequencing is a DNA sequence.

Sequence analyses were performed through a Basic Local Alignment Search Tool search using the nucleotide sequence databases available in the GenBank. Species determination may be made by a best match between the DNA sequence and a sequence in a database identifying an organism. In some cases, an additional confirmation is made by matching the species identified to a set of physical and biochemical characteristics known in the art as belonging to such particular organism. The set of characteristics for the identified species may be determined from techniques such as culture growth rate, culture morphology, pigmentation, staining, and biochemical tests.

Controls

The species used as controls were derived from the American Type Culture Collection (ATCC) depository. The species used in this example are: M. tuberculosis (ATCC) 25177, M. kansasii ATCC 12748, M. avium ATCC 25291, M. gordonae ATCC 14471, Staphylococcus epidermidis ATCC 12228, Streptococcus pneumoniae ATCC 49619, Pseudomonas aeruginosa ATCC 27853, Branhamella catarrhalis ATCC 25240, E coli ATCC 29194, and Neisseria lactamica ATCC 23970.

Identification Methods A comparison of a PCR method of the present invention, the biochemical identification method and the NAH probe method was performed for 69 unknown clinical specimens. The sources of the specimens were the respiratory tract, blood from a central venous catheter, and other bodily tissues or fluids. The probes used in the NAH method (Gen-probe Inc, San Diego, Calif.) included M. tuberculosis complex, M. avium-intracellulare complex, M gordonae, and M kansasii. Specimens that showed negative for NAH-probe were identified by biochemical methods (Metchock et al., 1997) or by a mycolic acid analysis by use of a high-performance liquid chromatography at another laboratory (Focus Technologies, Cypress, Calif.).

Results

Three methods of species determination were compared with the PCR method representing an embodiment of the present invention. Table 1 provides a summary of such comparison. The NAH-probe method yield an identification for 44 of the 69 unknowns species. Twenty-three of the 44 identified specimens provided a determination at the species level. The remaining 21 of the 44 specimens provided identification that is limited to the genus level or higher up the classification level. The biochemical and mycolic acid methods identified 68 of the 69 organisms at the species level. Table 1 summaries the remaining 25 NAH-negative specimens that were subjected to the biochemical or mycolic acid analysis. Most of the specimens were identified at the species level, with 3 specimens identified at the genus level or higher. The PCR method conclusively identified all 68 of the 69 specimens at the species level and one specimen at the genus level.

TABLE 1
Comparison of 16S rDNA PCR and traditional methods of
identification of Mycobacteria.

| | | | | | | |
|---|---|---|---|---|---|---|
| | Culture | ID by non- | Time | ID by 16S | | Time |
| N | time (D) | PCR methods | (D) | rDNA PCR | Time (D) | Saved (D) |

Identification by current NAH (n = 44)

| | | | | | | |
|---|---|---|---|---|---|---|
| 3 | 15.0 | MTB complex | 2.7 | M. tuberculosis/ | 2.7 | 0.0 |
| | | | | M. bovis | | |
| 4 | 32.8 | M. kansasii | 3.8 | M. kansasii | 3.0 | 0.8 |
| 19 | 29.5 | M. gordonae | 4.7 | M. gordonae | 2.8 | 1.9 |
| 9 | 25.8 | MAI complex | 4.6 | M. avium | 2.8 | 1.8 |
| 9 | 25.8 | MAI complex | 4.6 | M. intracellulare | 5.4 | −0.8 |
| Mean | 27.3 | | 4.5 | | 3.4 | 1.1 |

Identification by biochemical tests or Mycolic Acid Analysis (n = 25)

| | | | | | | |
|---|---|---|---|---|---|---|
| 5 | 9.4 | M. fortuitum | 69.2 | M. fortuitum | 4.0 | 65.2 |
| 2 | 10.5 | M. chelonae | 50.0 | M. chelonae | 2.5 | 47.5 |
| 1 | 5.0 | M. abscessus | 36.0 | M. abscessus | 2.0 | 34.0 |
| 1 | 10.0 | M. simiae | 66.0 | M. simiae | 5.0 | 61.0 |
| 1 | 43.0 | M. szulgai | 93.0 | M. szulgai | 2.0 | 91.0 |
| 1 | 7.0 | MCF complex | 44.0 | M. fortuitum | 4.0 | 40.0 |
| 4 | 7.8 | M. mucogenicum | 39.3 | M. mucogenicum | 3.3 | 36.0 |
| 1 | 11.0 | M. mucogenicum | 67.0 | M. chelonae/ | 4.0 | 63.0 |
| | | | | M. abscessus | | |
| 2 | 29.5 | M. simiae complex | 70.0 | M. lentiflavum | 3.5 | 66.5 |
| 2 | 9.5 | M. fortuitum | 46.5 | M. farcinogenes | 2.0 | 44.5 |
| 1 | 30.0 | M. scrofulaceum | 93.0 | M. paraffinicum | 2.0 | 91.0 |
| 1 | 19.0 | No ID | 107.0 | M. kubicae | 3.0 | 104.0 |
| 2 | 4.0 | MCF complex | 41.5 | Mycobacterium | 3.0 | 38.5 |
| | | | | species | | |
| 1 | 16.0 | M. fortuitum | 40.0 | M. szulgai | 5.0 | 35.0 |
| Mean | 15 | | 58.6 | | 3.3 | 55.3 |

The amount of time required for species identification by the NAH probe method, if an organism can be identified by this method, is not significantly longer than the time required for identification by the PCR method. The 16S rDNA sequencing method identified all organisms in a single method and was slightly faster by approximately 1 day. Specimens which could not be identified by the NAH method were subjected to biochemical tests and/or mycolic acid analysis. This two-step approach to organism identification significantly increase the turnaround time by approximately 55 days.

Therefore, the PCR method of species determination offers several distinct advantages, being species-specific level determination, reduce the need for a second or third method, and a rapid identification.

It should be understood, of course, that the forgoing disclosure relates to only an embodiment of the invention and the numerous modifications or alterations may be made therein without departing from the spirit and scope of the invention as set forth in the appended claims.

### ADVANTAGES AND INDUSTRIAL APPLICABILITY

It will be apparent from the description herein that an assay of the present invention has a number of advantages over previous known techniques used to identify species of Mycobacteria. For example, the present assay does not require the use of multiple primer sets, specific to each of the species of Mycobacteria which one wishes to detect, but rather a single genus-specific primer set may be utilized. Species identification may be performed by comparing the nucleotide sequence of a PCR product with a known nucleotide sequences. To provide another example, the assay does not required the use of complex amplification methods, such as real-time, nested, or semi-nested PCR methods. However, use of the present invention does not preclude such and other complex amplification methods. In some embodiments, use of complex PCR may be preferred.

The technique offers significant advantages over others in that it employs well-defined primers to specific regions of the nucleotide sequence for PCR at relatively high stringency, thereby decreasing contamination of the host DNA and with reproducibility as a consequence of non-specificity of primers and the low annealing temperature in PCR.

Taken together, the above novel features allow for the rapid, high resolution, qualitative screening of large number of samples for any species of the acid-fast bacterium. Further, the assay obviates the need to conduct following up biochemical tests, which reduces time, labor and expense.

The assay of the present invention may be used, experimentally or on a commercial scale, as a means of routine diagnosis and monitoring of acid-fact bacteria. Alternatively, the assay may be applicable to the quality control of species status of monospecific laboratory lines of Mycobacteria. It may be further useful as a complementary tool in the development of future commercial vaccines and diagnostic tests.

There is intraspecific and interspecific DNA sequence variation within species of acid-fast bacteria. As a result, prior techniques used to detect species of acid-fast bacteria have steered clear of utilizing anything but species-specific PCR so as to obviate the need for necessary extensive sequencing and characterization of resultant PCR products in order to identify isolates, at least at the species level. In contrast, the present invention utilizes genus-specific PCRs which specifically target variation in sequences both within and between species of Mycobacteria. This technique has surprisingly proven to be advantageous.

The invention described herein, with reference to certain preferred embodiments, in order to enable the reader to practice the invention without undue experimentation. However, a person having ordinary skill in the art will readily recognize that many of the components and parameters may be varied or modified to a certain extent without departing from the scope of the invention. Furthermore, title, headings, or the like are provided to enhance the reader's comprehension of this document, and should not be read as limiting the scope of the present invention.

### References

1. Alcaide F, Benitez M A, Escriba J M, et al. Evaluation of the BACTEC MGIT 960 and the MB/BacT systems for recovery of Mycobacteria from clinical specimens and for species identification by DNA AccuProbe. J Clin Microbiol. 2000; 38:398-401.
2. Williams-Bouyer N, Yorke R, Lee H I, et al. Comparison of the BACTEC MGIT 960 and ESP culture system II for growth and detection of Mycobacteria. J Clin Microbiol. 2000; 38:4167-4170.
3. Tortoli E, Cichero P, Piersimoni C, et al. Use of BACTEC MGIT 960 for recovery of Mycobacteria from clinical specimens: multicenter study. J Clin Microbiol. 1999; 37:3578-3582.
4. Hale Y M, Pfyffer G E, Salfinger M. Laboratory diagnosis of Mycobacterial infections: new tools and lessons learned. Clin Infect Dis. 2001; 33:834-846.
5. Turenne C Y, Tschetter L, Wolfe J. et al. Necessity of quality- controlled 16S rRNA gene sequence databases: identifying nontuberculous Mycobacterium species. J Clin Microbiol. 2001; 39:3637-3648.
6. Patel J B, Leonard D G, Pan X, et al. Sequence-based identification of Mycobacterium species using the Microseq 500 16S rDNA bacterial identification system. J Clin Microbiol. 2000; 38:246-251.
7. Cloud J L, Neal H, Rosenberry R, et al. Identification of Mycobacterium spp by using a commercial 16S ribosomal DNA sequencing kit and additional sequencing libraries. J Clin Microbiol. 2002; 40:400-406.
8. Kolbert C P, Persing D H. Ribosomal DNA sequencing as a tool for identification of bacterial pathogens. Curr Opin Microbiol. 1999; 2:299-305.
9. Drancourt M, Bollet C, Carlioz A, et al. 16S Ribosomal DNA sequence analysis of a large collection of environmental and clinical unidentifiable bacterial isolates. J Clin Microbiol. 2000; 38:3623-3630.
10. Rogall T, Flohr T, Bottger E C. Differentiation of Mycobacterium species by direct sequencing of amplified DNA. J Gen Microbiol. 1990; 136: 1915-1920.
11. Holberg-Petersen M, Steinbakk M, Figenschau K J, et al. Identification of clinical isolates of Mycobacterium spp by sequence analysis of the 16S ribosomal RNA gene: experience from a clinical laboratory. APMIS. 1999; 107:231-239.
12. Tortoli E, Bartoloni A, Böttger E C, et al. Burden of unidenti-fiable Mycobacteria in a reference laboratory. J Clin Microbiol. 2001; 39:4058-4065.
13. Metchock B G, Nolte F S, Wallace R J. Mycobacterium. In: Murray P R, Baron E J, Pfaller M A, et al, eds. Manual of Clinical Microbiology. 7th ed. Washington, D.C.: ASM Press; 1997:399-437.
14. Springer B, Bottger E C, Kirschner P, et al. Phylogeny of Mycobacterium chelonae-like organism based on partial sequencing of the 16S rRNA gene and proposal of Mycobacterium mucogenicum sp nov. Int J Syst Bacteriol. 1995; 45:262-267.
15. Springer B, Wu W K, Bodmer T, et al. Isolation and characterization of a unique group of slowly growing Mycobacteria: description of Mycobacterium lentiflavum sp nov. J Clin Microbiol. 1996; 34:1100-1107.
16. Floyd M M, Gross W M, Bonato D A, et al. Mycobacterium kubicae sp nov, a slowly growing, scotochromogenic Mycobacterium. Int J Syst Evol Microbiol. 2000; 50(50 pt 5):1811-1816.
17. Han X Y, Pham A S, Tarrand, J J, Sood, P K, Luthra R. Rapid and accurate identification of Mycobacteria by sequencing hypervariable regions of the 16S ribosomal RNA gene.
18. Karlin, S. & Altschul, S. F. (1990) Methods of assessing the statistical significance of molecular sequence features by using general scoring schemes. Proceeding Natl Aca. Sci. USA 1990, 87:2262-2268
19. Altschul, S. F., Gish, W., Miller, W., Myers, E. W. & Lipman, D. J. (1990) Basic Local Alignment Search Tool. J. Mol. Biol 215:403-410

1. A method for determining a bacterium species, comprising:
annealing a region of a nucleotide template to a specific oligonucleotide primer set comprising SEQ-FOR and SEQ-REV in a complimentary fashion, said primer set designed to provide a product having a predetermined size dictated by a complimentary primer set;
amplifying said region of said nucleotide template to produce said product;

2. The method of claim 1, wherein said SEQ-FOR is an AFB-f selected from SEQ ID NO: 18 through SEQ ID NO: 53 or a fragment or a variation thereof, and said SEQ-REV is an AFB-r selected from SEQ ID NO: 54 through SEQ ID NO: 89 or a fragment or a variation thereof.

3. The method of claim 1, wherein said SEQ-FOR is an UB-f selected from said SEQ ID NO: 90 through SEQ ID NO: 117 or a fragment or a variation thereof, and said SEQ-REV is an UB-r, a reverse primer sequence selected from said SEQ ID NO: 118 through SEQ ID NO: 145 or a fragment or a variation thereof.

4. The method of claim 1, wherein said SEQ-FOR is AFB-f selected from SEQ ID NO: 18 through said SEQ ID NO: 53 or a fragment or a variation thereof, and said SEQ-REV is an UB-r selected from SEQ ID NO: 118 through SEQ ID NO: 145 or a fragment or a variation thereof.

5. A method for determining a bacterium species, comprising:
annealing a region of a nucleotide template to a specific oligonucleotide primer set comprising SEQ-FOR and SEQ-REV in a complimentary fashion, said primer set designed to provide a product having a predetermined size dictated by a complimentary primer set;
amplifying said region of said nucleotide template to produce said product; and
determining a species of a bacterium in a nucleotide sequence of said product.

6. The method of claim 5, wherein annealing said region of said nucleotide template includes a hypervariable region of said nucleotide template.

7. The method of claim 5, wherein amplifying said region of said nucleotide template includes a polymerase chain reaction having at least approximately 15 cycles.

8. The method of claim 5, wherein said SEQ-FOR is an AFB-f selected from SEQ ID NO: 18 through SEQ ID NO: 53 or a fragment or a variation thereof, and said SEQ-REV is an AFB-R selected from SEQ ID NO: 54 through SEQ ID NO: 89 or a fragment or a variation thereof.

9. The method of claim 5, wherein said SEQ-FOR is an UB-f selected from said SEQ ID NO: 90 through SEQ ID NO: 117 or a fragment or a variation thereof, and said SEQ-REV is an UB-r, a reverse primer sequence selected from said SEQ ID NO: 118 through SEQ ID NO: 145 or a fragment or a variation thereof.

10. The method of claim 5, wherein said SEQ-FOR is AFB-f selected from SEQ ID NO: 18 through said SEQ ID NO: 53 or a fragment or a variation thereof, and said SEQ-REV is an UB-r selected from SEQ ID NO: 118 through SEQ ID NO: 145 or a fragment or a variation thereof.

11. The method of claim 8, wherein a length of said product is between approximately 550 base pairs and approximately 650 base pairs.

12. The method of claim 8 or 9, wherein a length of said product is between approximately 500 base pairs and approximately 700 base pairs.

13. The method of claim 10, wherein a length of said product is between approximately 1000 base pairs and 1700 base pairs.

14. The methods of claim 5, including comparing said product and said nucleotide sequence to determine said bacterium species to be a Mycobacterium, an acid-fast bacillus, or a nocardium.

15. The method of claim 5, further comprising:
(a) providing a query of said product to a BLAST™ program;
(b) substantially matching said query and a subject to provide a nucleotide identity; and
(c) determining said bacterium species using said nucleotide identity.

16. A method for determining a bacterium species, comprising:
extracting a genomic nucleotide from a specimen to provide a nucleotide template;
annealing a region in the nucleotide template to specific oligonucleotide primer set comprising SEQ-FOR and SEQ-REV in a complimentary fashion, said primer set designed to provide to a product having a particular size dictated by the complimentary primer set;
amplifying said region of said nucleotide template to produce said product; and
determining a species of a bacterium in a nucleotide sequence of said product.

17. The method of claim 16, wherein extracting said genomic nucleotide is performed using a PrepMan™ Reagent.

18. The method of claim 16, including selecting said specimen from blood, sputum, bronchial alveolar lavage, bronchial wash, tissue biopsy, body fluids, pus, urine, bone marrow aspirate, gastric aspirate, stool, tissue fluid, implanted medical device, prosthetic device, soil, sludge, water or liquids.

19. The method of claim 16, wherein said region of said nucleotide template is a hypervariable region.

20. The methods of claim 16, including comparing said product and said nucleotide sequence to determine said bacterium species to be a Mycobacterium, an acid-fast bacillus, or a nocardium.

21. The method of claim 16, further comprising:
(a) providing a query of said product to a BLAST™ program;
(b) substantially matching said query and a subject to provide a nucleotide identity; and
(c) determining said bacterium species using said nucleotide identity.

22. The method of claim 16, wherein said SEQ-FOR is an AFB-f selected from SEQ ID NO: 18 through SEQ ID NO: 53 or a fragment or a variation thereof, and said SEQ-REV is an AFB-r selected from SEQ ID NO: 54 through SEQ ID NO: 89 or a fragment or a variation thereof

23. The method of claim 16, wherein said SEQ-FOR is an UB-f selected from said SEQ ID NO: 90 through SEQ ID NO: 117 or a fragment or a variation thereof, and said SEQ-REV is an UB-r, a reverse primer sequence selected from said SEQ ID NO: 118 through SEQ ID NO: 145 or a fragment or a variation thereof.

24. The method of claim 16, wherein said SEQ-FOR is AFB-f selected from SEQ ID NO: 18 through said SEQ ID NO: 53 or a fragment or a variation thereof, and said SEQ-REV is an UB-r selected from SEQ ID NO: 118 through SEQ ID NO: 145 or a fragment or a variation thereof.

25. The method of claim 22, wherein a length of said product is between approximately 550 base pairs and approximately 650 base pairs.

26. The method of claim 22 or 23, wherein a length of said product is between approximately 500 base pairs and approximately 700 base pairs.

27. The method of claim 24, wherein a length of said product is between approximately 1000 base pairs and 1700 base pairs.

28. A method for determining a bacterium species, comprising:
culturing a bacterium from a specimen;
extracting genomic nucleotide from said bacterium to provide a nucleotide template;
annealing a desired region in the nucleotide template to a specific oligonucleotide primer set comprising SEQ-FOR and SEQ-REV in a complimentary fashion, said primer set designed to provide a product having a particular size dictated by the complimentary primer set;
amplifying said region of said nucleotide template to produce said product;
detecting said product; and
determining a species of a bacterium from a nucleotide sequence of said product.

29. The method of claim 28 including detecting said product by a gel electrophoresis and comparing said PCR product in an ethidium bromide stained-agarose gel to a DNA molecular weight ladder to determine a size of said PCR product.

30. The method of claim 28, wherein said genomic nucleotide is extracted using a PrepMan™ Reagent.

31. The method of claim 28, wherein annealing said region of said nucleotide template includes a hypervariable region of said nucleotide template.

32. The method of claim 28, wherein amplifying said region of said nucleotide template includes a polymerase chain reaction having at least approximately 15 cycles.

33. The methods of claim 28, including comparing said product and said nucleotide sequence to determine said bacterium species to be a Mycobacterium, an acid-fast bacillus, or a nocardium.

34. The method of claim 28, further comprising:
(a) providing a query of said product to a BLAST™ program;
(b) substantially matching said query and a subject to provide a nucleotide identity; and
(c) determining said bacterium species using said nucleotide identity.

35. The method of claim 28, wherein said SEQ-FOR is an AFB-f selected from SEQ ID NO: 18 through SEQ ID NO: 53 or a fragment or a variation thereof, and said SEQ-REV is an AFB-r selected from SEQ ID NO: 54 through SEQ ID NO: 89 or a fragment or a variation thereof.

36. The method of claim 28, wherein said SEQ-FOR is an UB-f selected from said SEQ ID NO: 90 through SEQ ID NO: 117 or a fragment or a variation thereof, and said SEQ-REV is an UB-r, a reverse primer sequence selected from said SEQ ID NO: 118 through SEQ ID NO: 145 or a fragment or a variation thereof.

37. The method of claim 28, wherein said SEQ-FOR is AFB-f selected from SEQ ID NO: 18 through said SEQ ID NO: 53 or a fragment or a variation thereof, and said SEQ-REV is an UB-r selected from SEQ ID NO: 118 through SEQ ID NO: 145 or a fragment or a variation thereof.

38. The method of claim 35, wherein a length of said product is between approximately 550 base pairs and approximately 650 base pairs.

39. The method of claim 35 or 36, wherein a length of said product is between approximately 500 base pairs and approximately 700 base pairs.

40. The method of claim 37, wherein a length of said product is between approximately 1000 base pairs and 1700 base pairs.

41. A method of determining bacterium species, comprising:
providing a specimen having a template;
providing a pair of primers selected from a group consisting of:
(a) a first forward primer having consecutive bases of an AFB-f selected from SEQ ID NO: 18 through SEQ ID NO: 53 or a fragment or a variation thereof and a first reverse primer having consecutive bases of an AFB-r selected from SEQ ID NO: 54 through SEQ ID NO: 89 or a fragment or a variation thereof;
(b) a second forward primer having consecutive bases of an UB-f selected from SEQ ID NO: 90 through SEQ ID NO: 117 or a fragment or a variation thereof and a second reverse primer having consecutive bases of an UB-r selected from SEQ ID NO: 118 through SEQ ID NO: 145 or a fragment or a variation thereof; and
(c) a first forward primer having consecutive bases of an AFB-f selected from SEQ ID NO: 18 through SEQ ID NO: 53 or a fragment or a variation thereof and a second reverse primer having consecutive bases of an UB-r selected from SEQ ID NO: 118 through SEQ ID NO: 145 or a fragment or a variation thereof;
amplifying a region of said template using said pair of primers to produce a product from said specimen; and
comparing said product from said specimen with a nucleotide sequence from a database to determine said bacterium species.

42. The method of claim 41, wherein said region of said template is a hypervariable region by which said product may be a species-specific nucleotide sequence derived from any combination of said PCR primers.

43. The method of claim 41, wherein said AFB-f and said AFB-R are specific for a bacterium including acid-fast bacillus by which said product may be a species-specific nucleotide sequence derived from said primers.

44. The method of claim 41, wherein said UB-f and said UB-r are universal primers for amplification a bacterium by which said product may be a species-specific nucleotide sequence derived from said primers.

45. A method of determining a bacterium species, comprising:
providing a sample having a template;
providing a forward primer having consecutive bases of an AFB-f selected from SEQ ID NO: 18 through SEQ ID NO: 53 or a fragment or a variation thereof and a reverse primer having consecutive bases of an AFB-r selected from SEQ ID NO: 54 through SEQ ID NO: 89 or a fragment or a variation thereof; and
amplifying a region of said template using said pair of primers to produce a product from said specimen; and
comparing said product from said specimen with a nucleotide sequence from a database to determine said bacterium species present in the specimen.

46. A method of determining a bacterium species, comprising:
providing a sample having a template;
providing a forward primer having consecutive bases of an UB-f selected from SEQ ID NO: 90 through SEQ ID NO: 117 or a fragment or a variation thereof and a reverse primer having consecutive bases of an UB-r selected from SEQ ID NO: 118 through SEQ ID NO: 145 or a fragment or a variation thereof;
amplifying a region of said template using said forward primer or said reverse primer to produce a product from said specimen; and
comparing said a product from said specimen with a nucleotide sequence from databases to determine said bacterium species present in the specimen.
